(19) United States
(12) Patent Application Publication
(10) Pub. No.: US 2019/0000672 A1
(43) Pub. Date: Jan. 3, 2019

(54) SYSTEMS AND METHODS FOR ACTUATING A VITRECTOMY PROBE USING A FLUIDIC AMPLIFIER OR OSCILLATOR

(71) Applicant: NOVARTIS AG, Basel (CH)

(72) Inventor: Brian William McDonell, Irvine, CA (US)

(21) Appl. No.: 16/008,708

(22) Filed: Jun. 14, 2018

Related U.S. Application Data
(60) Provisional application No. 62/526,174, filed on Jun. 28, 2017.

(51) Int. Cl. A61F 9/007

(52) U.S. Cl. CPC A61F 9/00763; A61F 9/00745

(57) ABSTRACT

The present disclosure discloses systems and methods for actuating a cutter of a vitrectomy probe. The systems and methods involve applying fluidic pressure to a fluidic amplifier or a fluidic oscillator of the vitrectomy probe to cause oscillatory movement of a component of the cutter to perform a cutting action.

### TECHNICAL FIELD

The present disclosure relates to ophthalmic surgery and surgical equipment and, more specifically, to systems and methods for actuating an inner cutter of a vitrectomy probe.

### BACKGROUND

Ophthalmic surgery saves and improves the vision of tens of thousands of patients every year. However, given the sensitivity of vision to even small changes in the eye and the minute and delicate nature of many eye structures, ophthalmic surgery is difficult to perform and the reduction of even minor or uncommon surgical errors or modest improvements in accuracy of surgical techniques can make an enormous difference in the patient's vision after the surgery.

Ophthalmic surgery is surgery performed on the eye or any part of the eye. Ophthalmic surgery is regularly performed, for example, to repair retinal defects, repair eye muscles, remove cataracts or cancer, or to restore or improve vision. Vitreous humor or vitreous is contained in the posterior segment of the eye and is a clear gel-like substance that holds the retina in place. In some surgical procedures, a surgeon may need to remove some or all of the vitreous to allow access to the retina or other internal structures. In such procedures, the vitreous may be cut and aspirated out of the eye in a procedure called a vitrectomy.

### SUMMARY

According to one aspect, the present disclosure describes a vitrectomy probe including a body defining a first diaphragm chamber; a cutter including tubular outer cutter coupled at a proximal end of the body; and a tubular inner cutter disposed within the tubular outer cutter and movable therewithin; and an actuating mechanism. The actuating mechanism includes a flexible diaphragm coupled to the body, the first diaphragm chamber disposed adjacent to a first side of the flexible diaphragm; and a fluidic amplifier. The fluidic amplifier includes an interaction region; a supply port in fluid communication with the interaction region; a first control port in fluid communication with the interaction region; a vent port through which fluid is vented from the fluidic amplifier; a splitter disposed at a distal end the interaction region and dividing the distal end of the interaction region into a first outlet and a second outlet; and a first vent line fluidically coupled to the first outlet of the interaction region and fluidically coupled to the vent port. The supply port may be operable to introduce a power stream of fluid into the interaction region. The first control port may be operable to introduce a first control jet of fluid into the interaction region. The first outlet may be fluidically coupled to the first diaphragm chamber and the second outlet fluidically coupled to the vent port.

Another aspect of the disclosure encompasses a method for actuating a cutter of a vitrectomy probe including supplying a power stream to a fluidic amplifier disposed within the vitrectomy probe. A tubular inner cutter of the cutter may be disposed in a first position when the power stream is supplied to the fluidic amplifier. A control jet may be selectively supplied to the fluidic amplifier with such that when the control jet is supplied. The power stream may be redirected from a first path to a second within the fluidic amplifier to actuate the tubular inner cutter from the first position to a second position. When the control jet is not supplied, the power stream may be returned to the first path within the fluidic amplifier, causing the tubular inner cutter to return to the first position.

Another aspect of the disclosure encompasses a method for actuating a tubular inner cutter of a vitrectomy probe including supplying a power stream to a fluidic amplifier disposed within the vitrectomy probe, a tubular inner cutter of the cutter disposed in a first position when the power stream is supplied to the fluidic amplifier and selectively supplying a control jet to the fluidic amplifier such that: when the control jet is supplied, the power stream is redirected from a first path to a second within the fluidic amplifier to actuate the tubular inner cutter from the first position to a second position, and when the control jet is not supplied, the power stream is returned to the first path within the fluidic amplifier, causing the tubular inner cutter to return to the first position.

A further aspect of the disclosure encompasses a vitrectomy probe including a body defining a first diaphragm chamber; a tubular outer cutter coupled at a proximal end to the body; an aspiration port formed in a distal end of the tubular outer cutter; a tubular inner cutter disposed within the tubular outer cutter, the tubular inner cutter movable within the tubular outer cutter; and an actuating mechanism operable to actuate the tubular inner cutter. The actuating mechanism may be housed in the body and include a flexible diaphragm coupled to the body and a fluidic oscillator. The first diaphragm chamber may be disposed adjacent to a first side of the flexible diaphragm. The fluidic oscillator may include an interaction region; a supply port in fluid communication with the interaction region, the supply port operable to introduce a power stream of fluid into the interaction region; a vent port through which fluid is vented from the fluidic oscillator; a first feedback channel offset from the interaction region by a first wall, the first feedback channel operable to redirect the power stream of fluid in a first direction; a second feedback channel offset from the interaction region by a second wall, the second feedback channel operable to redirect the power stream in a second direction opposite the first direction; a nozzle at the distal end of the interaction region; a first outlet extending from the nozzle and fluidically coupled to the first diaphragm chamber; a second outlet extending from the nozzle and fluidically coupled to the vent port; and a first vent line fluidically coupled to the first outlet of the fluidic oscillator and fluidically coupled to the vent port.

Another aspect of the disclosure includes a method for actuating a cutter of a vitrectomy probe including supplying a power stream to a fluidic oscillator disposed within the vitrectomy probe, a tubular inner cutter of the cutter disposed in a first position when the power stream is supplied to the fluidic oscillator; and configuring the fluidic oscillator such that the power stream interacts with a first feedback channel and a second feedback channel so that the power stream oscillates and is redirected to actuate the tubular inner cutter between the first position and a second position.

The various aspects may include one or more of the following features. A spring may abut the flexible diaphragm along a second side of the flexible diaphragm opposite the first side. The splitter may be offset from the supply port such that the splitter redirects a flow from the supply port into the first output port. The splitter may be offset from the supply port such that the splitter redirects a flow from the supply port into the second outlet. The splitter may be offset from the supply port such that the splitter redirects a flow from the supply port into the first outlet. The interaction region may include a sidewall shaped to promote attachment of the power stream to the sidewall. The splitter is aligned with the supply port such that the splitter redirects a flow from the supply port substantially equally into the first outlet and the second outlet. A second diaphragm chamber may be disposed adjacent to a second side of the flexible diaphragm. The second outlet may be fluidically coupled to the second diaphragm chamber. A second vent line may extend from the second outlet to the vent port. The splitter may be offset from the supply port such that the splitter redirects a flow from the supply port into the first outlet of the interaction region. The interaction region may include a sidewall shaped to promote attachment of the power stream to the sidewall. A second control port may be fluidically coupled to the interaction region, the second control portion operable to introduce a second control jet into the interaction region. The splitter may be aligned with the supply port such that the splitter divides a power stream flow from the supply port substantially equally into the first outlet of the interaction region and the second outlet of the interaction region.

The various aspects may include one or more of the following features. Actuation a cutter of a vitrectomy probe may also include determining a desired cutting rate of the cutter; configuring the vitrectomy probe and the fluidic amplifier such that the desired cutting rate for the vitrectomy probe is determined by and corresponds to a desired frequency of the control jet that is supplied to the fluidic amplifier; setting the control jet to a desired frequency, thereby setting the desired cutting rate for the vitrectomy probe; and supplying the control jet with a selected pressure so that the tubular inner cutter is actuated to either the first position or the second position. Actuation of a cutter of a vitrectomy probe may also include determining a desired cutting rate of the cutter; configuring the fluidic oscillator such that the desired cutting rate for the cutter corresponds to a desired frequency of the power stream oscillation; setting the desired cutting rate of the cutter by setting the power stream to a desired pressure level to produce the desired frequency of the power stream oscillation; and supplying the power stream at a selected pressure so that the tubular inner cutter oscillates at the desired frequency.

The various aspects may also include one or more of the following features. A second diaphragm chamber may be disposed on a second side of the flexible diaphragm opposite the first side. A second vent line may extend from the second outlet to the vent port. The first outlet of the fluidic oscillator may be fluidically coupled to one of the first diaphragm chamber or the second diaphragm chamber. The second outlet of the fluidic oscillator may be fluidically coupled to the other of the first diaphragm chamber or the second diaphragm chamber. A spring may abut the flexible diaphragm along a second side of the flexible diaphragm opposite the first side. The fluidic oscillator may also include a splitter that separates the first outlet from the second outlet.

The above systems and/or apparatuses may be used with the above methods and vice versa. In addition, any system or apparatus described herein may be used with any method described herein and vice versa. It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

For a more complete understanding of the present disclosure and its features and advantages, reference is now made to the following description, taken in conjunction with the accompanying drawings, which are not to scale and in which like numerals refer to like features.

FIG. 1 shows an example system for performing a vitrectomy including a schematic representation of a system for actuating an inner cutter of a vitrectomy probe.

FIG. 2 is a schematic representation of an example vitrectomy probe incorporating a fluidic amplifier within a double acting actuating mechanism;

FIG. 3 is a schematic representation of another example vitrectomy probe incorporating a fluidic amplifier within a double-acting actuating mechanism.

FIG. 4 is a schematic representation of still another vitrectomy probe incorporating a fluidic oscillator within a double-acting actuating mechanism.

FIG. 5 is a schematic representation of an example vitrectomy probe incorporating a fluidic amplifier within a single-acting actuating mechanism.

FIG. 6 is a schematic representation of a vitrectomy probe incorporating a fluidic oscillator within a single-acting actuating mechanism.

FIGS. 7-11 are schematic representations of various example fluidic amplifiers.

FIG. 12 is a schematic representation of an example fluidic oscillator.

FIGS. 13-15 are schematic representations of various example fluidic amplifiers.

FIG. 16 is a schematic representation of another example fluidic oscillator.

FIG. 17 is a flowchart of an example method for actuating an inner cutter of a vitrectomy probe.

FIG. 18 is a flowchart of another example method for actuating an inner cutter of a vitrectomy probe.

### DETAILED DESCRIPTION

In the following description, details are set forth by way of example to facilitate an understanding of the disclosed subject matter. It should be apparent to a person of ordinary skill in the field, however, that the disclosed embodiments are exemplary and not exhaustive of all possible embodiments. Thus, it should be understood that reference to the described example is not intended to limit the scope of the disclosure. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure.

The present disclosure provides systems and methods for actuating an inner cutting portion of a vitrectomy probe. The systems and methods use a fluidic amplifier or a fluidic oscillator within a vitrectomy probe to produce a pressure profile that is exerted on a flexible diaphragm within an actuating mechanism. A fluidic oscillator is a fluidic device that resonates in response to a constant fluidic input. A fluidic amplifier theoretically produces fluidic output in response to a constant fluidic input. A power stream is supplied to the amplifier by a pressure source. One or more control jets is supplied to the amplifier to drive the amplifier between various configurations. When the amplifier is in these various configurations the pressure profiles cause the diaphragm to actuate an inner cutter of a vitrectomy probe. A power stream is supplied to the oscillator by a pressure source. The power stream begins to oscillate as feedback currents are generated by the power stream's interaction with the internal structures of the fluidic oscillator, which are described below in detail. These oscillations cause pressure profiles that cause the diaphragm to actuate the inner cutter of a vitrectomy probe.

In a vitrectomy, the surgeon inserts small surgical instruments into the eye, such as a vitrectomy probe. Vitrectomy probes typically include a cutter that includes a hollow outer cutter and a hollow inner cutter. The inner cutter is arranged coaxially with and movably disposed within the hollow outer cutter. The cutter also includes an aspiration port formed in the outer cutter near the distal end thereof. Vitreous is aspirated into the open aspiration port, and the inner cutter is actuated and moved past the aspiration port, effectively closing the aspiration port. When the aspiration port closes, a cutting surface formed on a distal end of the inner cutter (generally a distal edge of the inner cutter) cooperates with a cutting edge of the aspiration port to cut the vitreous. In some instances, a distal edge and a proximal edge of the aspiration port may form cutting edges for severing vitreous. The severed vitreous is then aspirated away through the inner cutter. Some vitrectomy probes may also include a second aspiration port formed in the inner cutter near the distal end thereof. This second aspiration port includes a cutting edge and allows for vitreous to be aspirated, cut, and aspirated away twice during a single cycle of actuation for the vitrectomy probe.

The inner cutter may be actuated using various methods and actuating mechanisms. For example, the inner cutter may be moved from a proximal position to a distal position by applying sufficient pressure against a piston or diaphragm assembly to overcome a mechanical spring. When the pressure is reduced below a given threshold, the spring returns the inner cutter from the distal position to the proximal position. Alternatively, the inner cutter may be moved by creating a pressure differential across a piston or diaphragm assembly. The inner cutter may be placed in the distal position using a first source of pressure. The inner cutter may then be moved to the proximal position using a second source of pressure. Although pressure may exist on both sides of the piston or diaphragm assembly, creating a sufficient pressure differential will actuate the inner cutter.

In many conventional vitrectomy probes, the pressure used to actuate the inner cutter is controlled at a remote console. Long drive line tubes add resistance, i.e., head loss, and volume to the pressure control system. This arrangement reduces the ability to produce rapid changes in the actuator pressures, thereby limiting the cutting rate that can be achieved by the vitrectomy probe. Operating at higher cutting rates reduces the aspiration time between cuts, the turbulence of vitreous, and traction during cutting, which in turn helps prevent retinal injury or surgical complications that result from vitreous cutting.

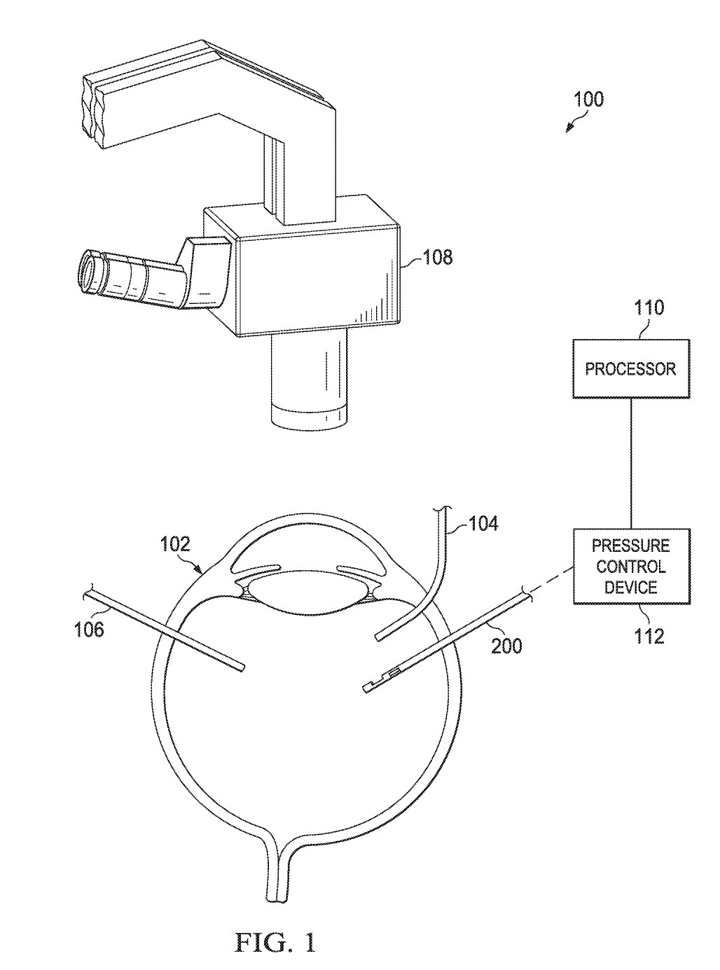

Referring now to the figures, FIG. 1 is a system 100 for actuating an inner cutter of a vitrectomy probe. As shown, the system 100 provides a vitrectomy probe 200 inserted into an eye 102. The eye 102 is not part of the system 100, but is shown to better illustrate how the system may be used. The system 100 also may include other surgical instruments 104 and 106, which may be, for example, an illuminator, an aspirator, or an infusion cannula. The system 100 also includes a processor 110 and a pressure control device 112. In some implementations, the processor 110 and the pressure control device 112 may be located in or otherwise form a part of a surgical console. The surgical microscope 108 is not part of the system 100, but the surgical microscope 108 is shown to better illustrate how the system 100 may be used.

As shown, during a vitrectomy, the vitrectomy probe 200 and the other surgical instruments 104 and 106 are inserted into the eye 102. The surgical microscope 108 is used to observe the vitrectomy and the surgical instruments 104 and 106 in the eye 102. The processor 110 is configured to communicate with the pressure control device 112 to control the intraocular pressure within the eye, such as by, for example, controlling the infusion pressure of fluid (e.g., a balanced salt solution) delivered to the eye and an aspiration pressure associated with withdrawing materials from the eye. Consequently, the pressure control device 112 is operable to control the aspiration pressure applied to the vitrectomy probe 200. In some implementations, the processor 110 and the pressure control device 112 are configured to control, for example, a power stream, a control jet or jets, a pressure level for both the power stream and the control jet(s), and the desired cutting rate of the vitrectomy probe. The power stream, the one or more control jets, and associated pressures thereof are described in more detail below.

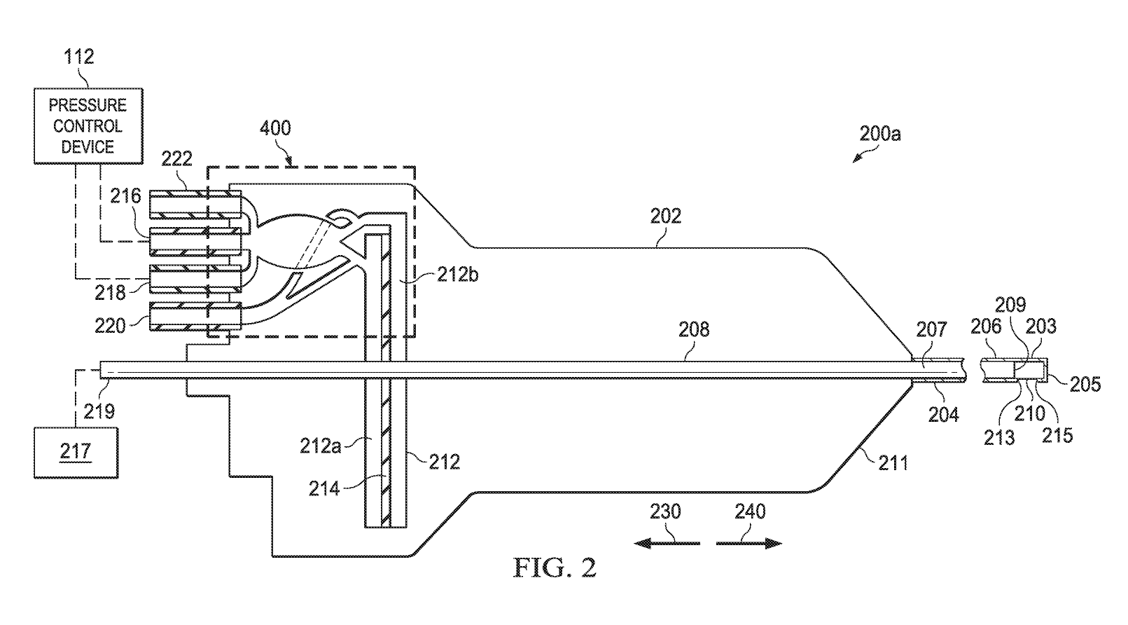

The pressure control device 112 is fluidically coupled to the vitrectomy probe 200, as shown in more detail in FIG. 2 through FIG. 6. Fluid that passes through the vitrectomy probes as described herein may be any fluid suitable to actuate the inner cutter. For instance, the fluid may be air or a liquid, particularly a liquid safe for use in a surgical setting, such as water or saline. For example, a balanced salt solution, such as BSS® or BSS PLUS® produced by Alcon Laboratories, Inc., located at 6201 South Freeway, Fort Worth, Tex. 76134, may be used. The pressure control device 112 may be configured with a spool or poppet pneumatic valve or with a high speed pneumatic valve (referred to collectively hereinafter as “high speed valve”). U.S. patent application Ser. No. 15/091686 (Publication No. 2016-0296370 A1), the entire contents of which are incorporated herein by reference, describes examples of a high speed pneumatic valve. The high speed pneumatic valves described in application Ser. No. 15/091686 allow for rapid transition between fluid supply and fluid exhaust. The high speed pneumatic valves continuously rotate in a single axial direction and rapidly positions ports of an instrument to be in fluid communication with either a fluid supply or a fluid exhaust.

The power stream pressure level may be based on the characteristics of the diaphragm that is used in the actuating mechanism of the vitrectomy probe. The power stream pressure level may also be set in relation to the level of the control jet pressure, and vice versa. The control jet pressure level may be based on the characteristics of the diaphragm that is used in the actuating mechanism of the vitrectomy probe. The control jet pressure level may also be set in relation to the level of the power stream pressure. The control jet pressure level may be smaller in magnitude than the power stream pressure level. Depending on the stage of the vitrectomy surgery, the user may desire a specific cutting rate of the vitrectomy probe. The cutting rate may correlate with the frequency of changes in the control jet pressure. The desired cutting rate of the vitrectomy probe may be defined by the user and one or multiple cutting rates may be used throughout the vitrectomy surgery. The user may select a desired cutting rate and duty cycle. Each power stream pressure level and each control jet pressure level may be determined by the processor 110, and one or multiple power stream pressures and control jet pressures may be used throughout the vitrectomy surgery.

The processor 110 may include, for example a microprocessor, microcontroller, digital signal processor (DSP), application specific integrated circuit (ASIC), or any other digital or analog circuitry configured to interpret and/or execute program instructions and/or process data. In some embodiments, the processor 110 may interpret and/or execute program instructions and/or process data stored in a memory. The memory may be configured in part or whole as application memory, system memory, or both. The memory may include any system, device, or apparatus configured to hold and/or house one or more memory modules. Each memory module may include any system, device, or apparatus configured to retain program instructions and/or data for a period of time (e.g., computer-readable media). The various servers, electronic devices, or other machines described may contain one or more similar such processors or memories for storing and executing program instructions for carrying out the functionality of the associated machine.

FIG. 2 is a schematic representation of an example vitrectomy probe 200a. As shown, vitrectomy probe 200a has a body 202 and a cutter 203. The cutter 203 includes an outer cutter 206, an inner cutter 208 and an aspiration port 210 formed in the outer cutter 206. The outer cutter 206 is attached at a proximal end 204 to a distal end 211 of the body 202 and includes a closed distal end 205. The inner cutter 208 is positioned concentrically within the outer cutter 206 as well as within and through the body 202. The inner cutter 208 is hollow to form a passage 207 and open at both the distal end 209 and proximal end 219. The distal end 209 of the inner cutter 208 includes a cutting surface to allow for tissue to be cut during actuation of the cutter 203. The aspiration port 210 is positioned toward the distal end 205 of the outer cutter 206 and extends radially through the outer cutter 206. The distal end 215 and proximal end 213 of the aspiration port 210 may have cutting surfaces to allow for tissue to be cut during actuation. The one or more cutting surfaces of the aspiration port 210 cooperate with the cutting surface of the inner cutter 208 to sever material that is drawn into the cutter 203 via the aspiration port 210. The severed material is then aspirated away through the passage 207 in the inner cutter 208.

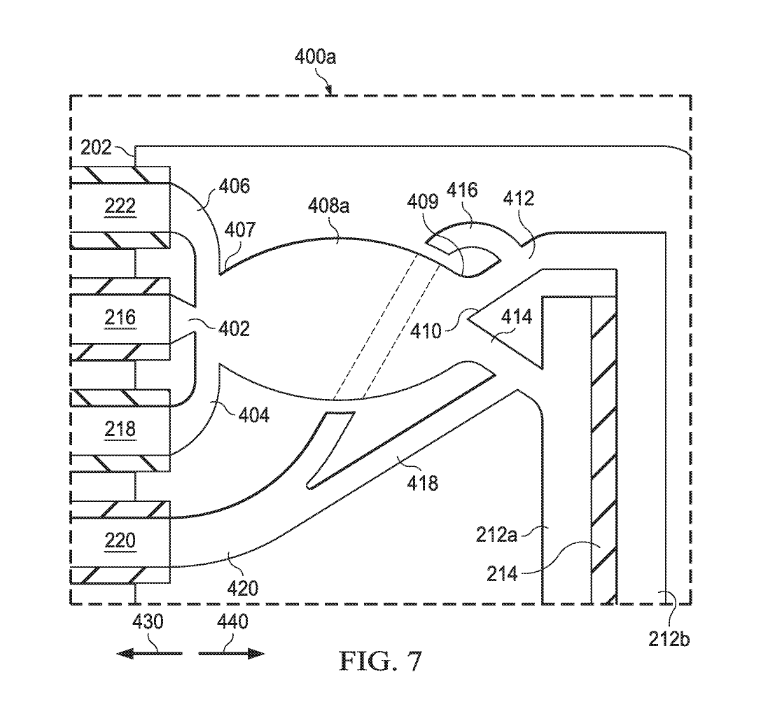

Within the body 202, the vitrectomy probe 200a also has a diaphragm chamber 212 that is divided into a proximal diaphragm chamber 212a and a distal diaphragm chamber 212b. The proximal diaphragm chamber 212a and the distal diaphragm chamber 212b are separated by a flexible diaphragm 214. The flexible diaphragm 214 may be coupled to the body 202 along an outer periphery thereof. The vitrectomy probe 200a also includes a fluidic amplifier 400, a power stream port 216, a first control jet port 218, a vent port 220, and a second control jet port 222. As shown in FIG. 2, the fluidic amplifier 400 is a general representation of various example fluidic amplifiers that are further described in reference to FIG. 7 through FIG. 9.

In some implementations, the inner cutter 208 is rigidly coupled to the flexible diaphragm 214 such that, when the flexible diaphragm 214 experiences a pressure differential between distal diaphragm chamber 212b and proximal diaphragm chamber 212a that is sufficient to displace the flexible diaphragm 214 (e.g., a pressure differential that overcomes the frictional resistance and inertia of the moving parts of the vitrectomy probe 200a), the flexible diaphragm 214 is displaced, thus actuating the inner cutter 208. When oscillatory pressure differentials are created between the distal diaphragm chamber 212b and the proximal diaphragm chamber 212a, the inner cutter 208 is actuated in a reciprocal manner at a desired cutting rate. The cutting rate of the inner cutter 208 and, hence, the cutter 203, varies in response to the rate of oscillation of the pressure differentials within the proximal diaphragm chamber 212a and the distal diaphragm chamber 212b.

When the pressure in the proximal diaphragm chamber 212a is greater than the pressure in the distal diaphragm chamber 212b, the inner cutter 208 is actuated in the direction of the arrow 240. As the inner cutter 208 is actuated, the distal end 209 of the inner cutter 208 also moves in the direction of the arrow 240 from a position proximal to the aspiration port 210 to a position distal of the aspiration port 210. When the pressure in the distal diaphragm chamber 212b is sufficiently greater than the pressure in proximal diaphragm chamber 212a so as to cause the inner cutter 208 to move (e.g., a pressure differential between the distal diaphragm chamber 212b and proximal diaphragm chamber 212a such that the pressure overcomes the frictional resistance and inertia of the moving parts of the vitrectomy probe 200a), the inner cutter 208 is actuated in the direction of the arrow 230. As a result, the inner cutter 208 is returned to its initial position, passing from the position distal to the aspiration port 210 to the position proximal to the aspiration port 210. In some instances, the vitrectomy probe 200a may include a mechanism to adjust the proximal position of the inner cutter 208 relative to the aspiration port 210, and this proximal position may be varied by a user, such as a surgeon, before, during, or after a surgical procedure.

When the inner cutter 208 is in the proximal position, vacuum pressure applied to the passage 207 draws tissue (e.g., vitreous) or other materials into the aspiration port 210. The vacuum pressure may be applied continuously or intermittently. When the inner cutter 208 is in the distal position, the cutting surfaces on the inner cutter 208 and the aspiration port 210 formed in the outer cutter 206 cut the tissue that has been drawn into the aspiration port 210. The vacuum pressure then aspirates the cut tissue and other materials through the passage 207 formed in the inner cutter 208 in the direction of the arrow 230. The aspirated materials may be collected in a collection chamber 217. The inner cutter 208 is then actuated in the direction of the arrow 230, returning the inner cutter 208 to the proximal position to allow for further aspiration.

In some implementations, the inner cutter 208 may also have a port formed therein proximal to the distal end 209. A distal end of the port formed in the inner cutter 208 may have one or more cutting surfaces to allow for tissue to be cut during actuation. Thus, in some implementations, the port formed in the inner cutter 208, in combination with the aspiration port 210, allows the vitrectomy probe 200a to perform two cuts during each actuation cycle of the inner cutter 208.

The power stream port 216, the first control jet port 218, the vent port 220, and the second control jet port 222 are each fluidically coupled to the pressure control device 112. As shown in FIG. 2, the power stream port 216, the first control jet port 218, the vent port 220, and the second control jet port 222 are also fluidically coupled to the fluidic amplifier 400. The pressure control device 112 supplies fluid to the vitrectomy probe 200a to actuate the inner cutter 208. The processor 110, shown in FIG. 1, and the pressure control device 112 regulate parameters, for example, pressure level and cycle frequency, of the power stream and the control jets.

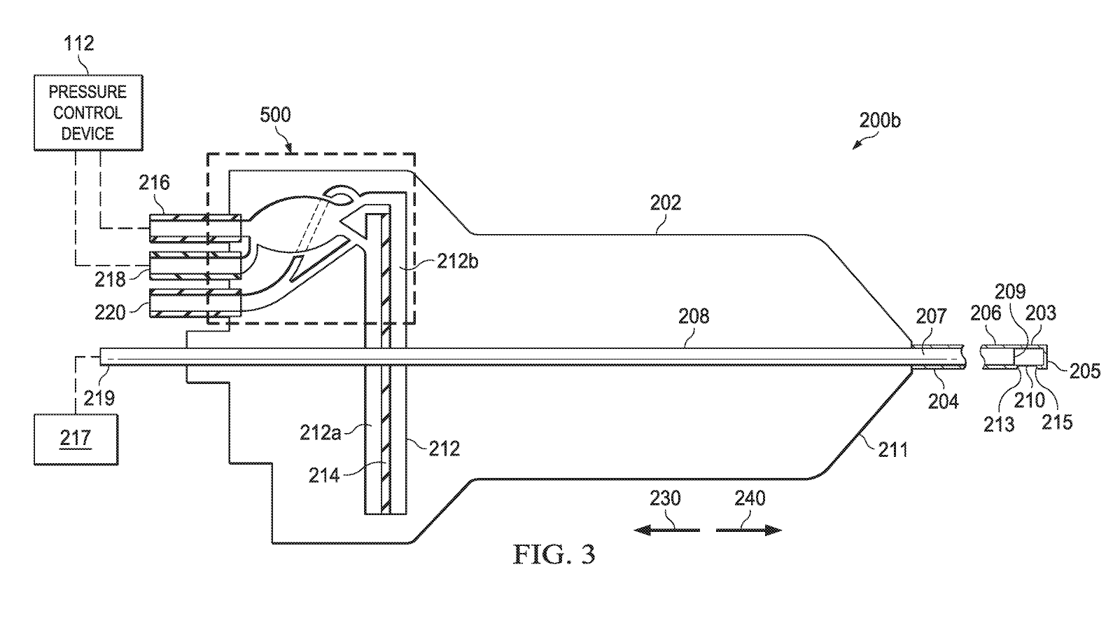

FIG. 3 is a schematic representation of an example vitrectomy probe 200b. The vitrectomy probe 200b is similar to the vitrectomy probe 200a as described above in reference to FIG. 2. In particular, the body 202, the cutter 203, the outer cutter 206, the passage 207, the inner cutter 208, the aspiration port 210, the diaphragm chamber 212, and the flexible diaphragm 214 all function and interact in the same way as described above in reference to FIG. 2.

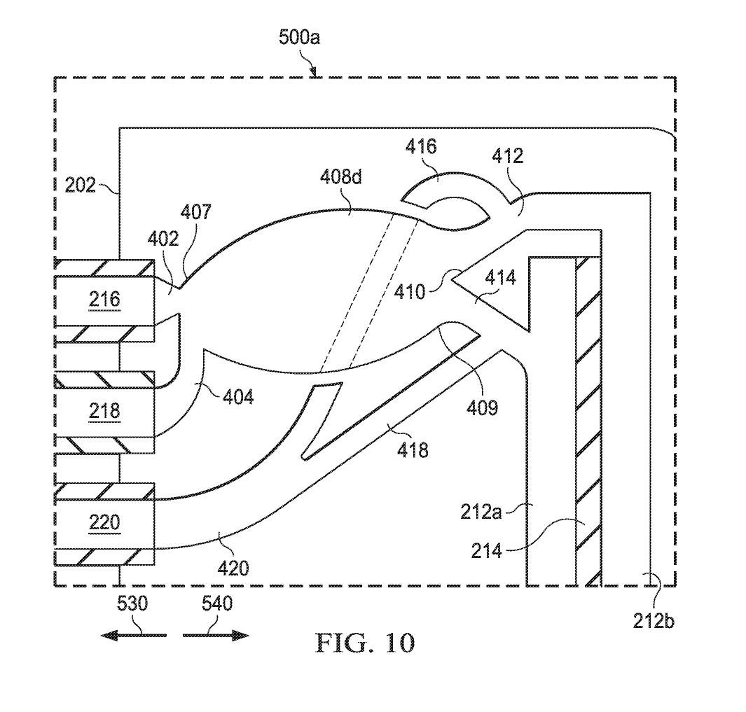

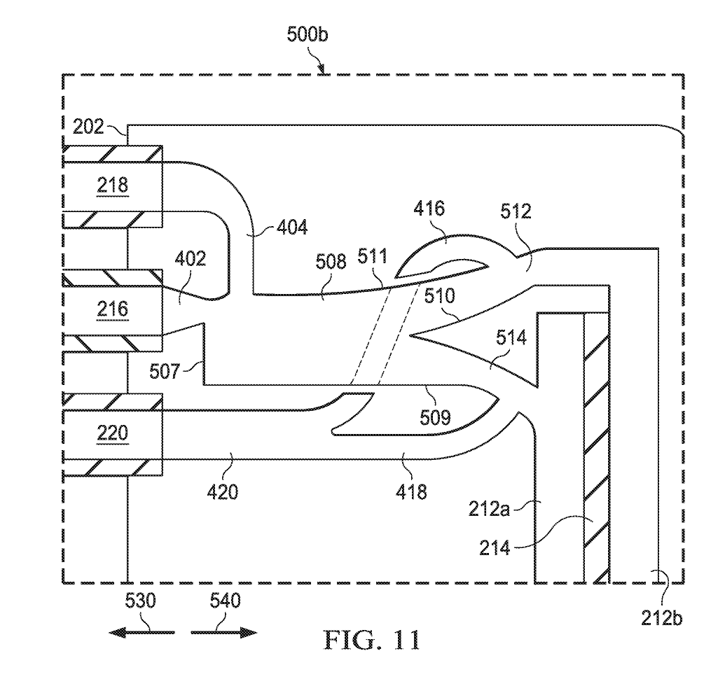

The vitrectomy probe 200b also includes a fluidic amplifier 500, a power stream port 216, a control jet port 218, and a vent port 220. As shown in FIG. 3, the fluidic amplifier 500 is a general representation of various example fluidic amplifiers that are further described in reference to FIGS. 10 and 11. FIG. 3 illustrates a vitrectomy probe 200b with a single control jet port 218. The fluidic amplifier 500 included within the vitrectomy probe 200b is configured to operate with the single control jet port 218 as opposed to the two control jet ports included within the vitrectomy probe 200a. Otherwise the control jet port 218, the power stream port 216, and the vent port 220 operate in the same way as already described above in reference to FIG. 2. In FIG. 3, the control jet port 218, the power stream port 216, and the vent port 220 are all fluidically coupled to the fluidic amplifier 500.

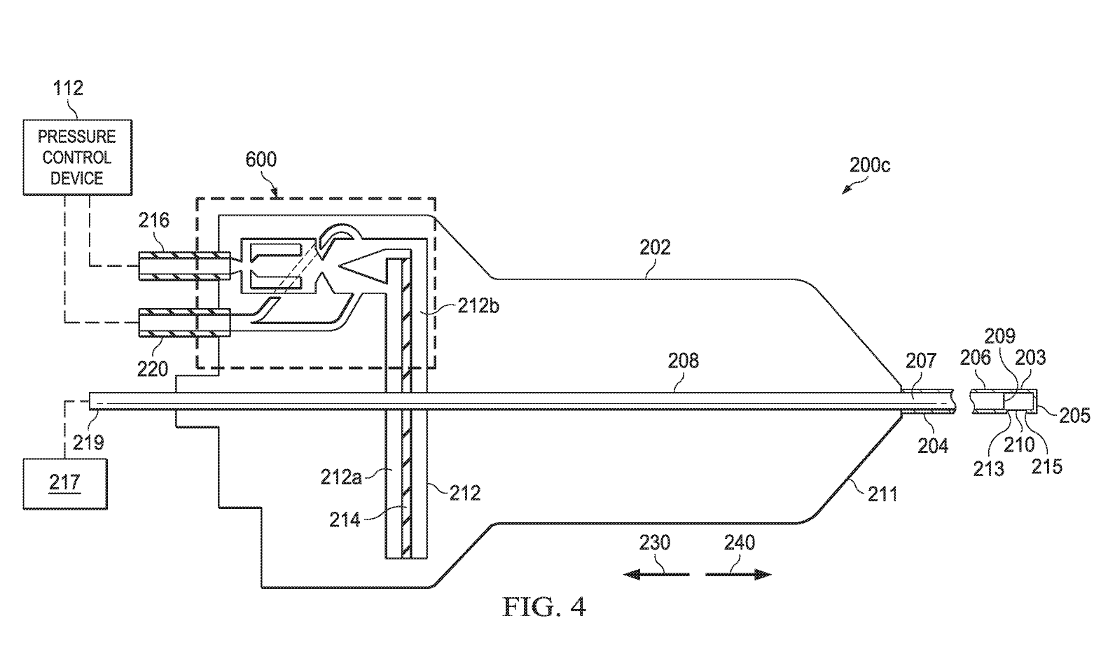

FIG. 4 is a schematic representation of another example vitrectomy probe 200c. The vitrectomy probe 200c is similar to the vitrectomy probes 200a and 200b shown in FIGS. 2 and 3, respectively. In particular, the body 202, the cutter 203, the outer cutter 206, the passage 207, the inner cutter 208, the aspiration port 210, the diaphragm chamber 212, and the flexible diaphragm 214 all function and interact in the same way as described above in reference to FIGS. 2 and 3.

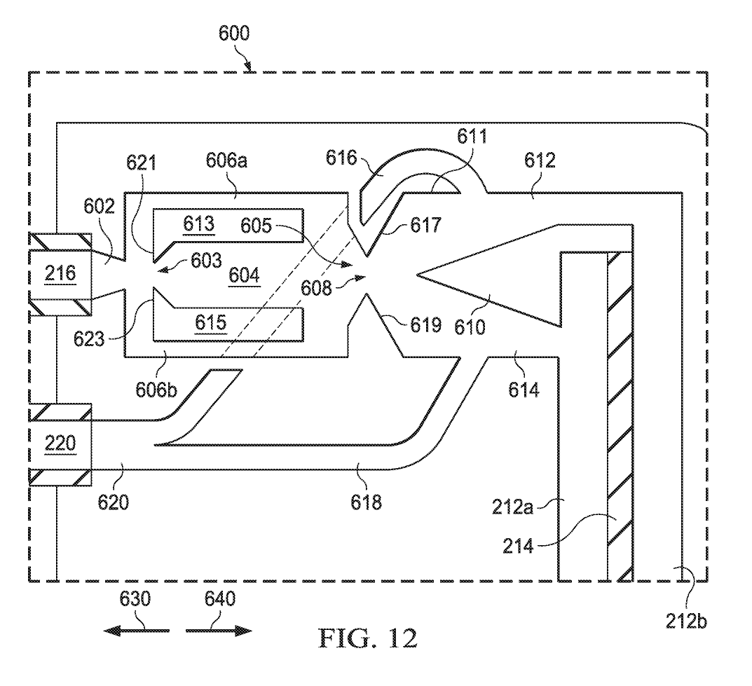

The vitrectomy probe 200c also includes a fluidic oscillator 600, a power stream port 216, and a vent port 220. As shown in FIG. 4, the fluidic oscillator 600 is a general representation of an example fluidic oscillator that is further described in reference to FIG. 12. FIG. 4 illustrates a vitrectomy probe 200c that does not require a control jet port to function. The fluidic oscillator 600 included within the vitrectomy probe 200c is configured to operate without the need for a control jet port. Otherwise the power stream port 216 and the vent port 220 operate in the same way as already described above in reference to FIG. 2. In FIG. 4, the power stream port 216 and the vent port 220 are fluidically coupled to the fluidic oscillator 600.

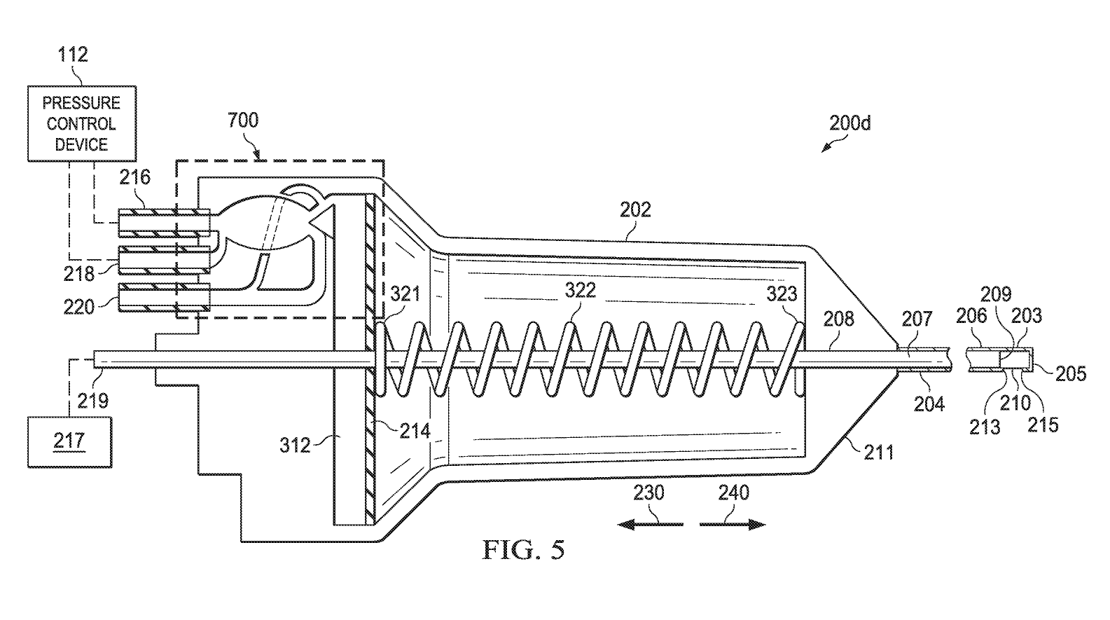

FIG. 5 is a schematic representation of a further example vitrectomy probe 200d. The vitrectomy probe 200d is similar to the vitrectomy probes 200a, 200b, and 200c shown in FIGS. 2, 3, and 4, respectively. In particular, the body 202, the cutter 203, the outer cutter 206, the passage 207, the inner cutter 208, and the aspiration port 210 all function and interact in the same way as described above in reference to FIGS. 2, 3, and 4.

Within the body 202, the vitrectomy probe 200d also includes a diaphragm chamber 312 and a flexible diaphragm 214. The diaphragm chamber 312 is disposed proximal and adjacent to the flexible diaphragm 214. The vitrectomy probe 200d also includes a fluidic amplifier 700, a power stream port 216, a control jet port 218, and a vent port 220. The vitrectomy probe 200d also includes a spring, such as a coil spring 322. The inner cutter 208 extends through the center of the coil spring 322. In some implementations, a proximal end 321 of the coil spring 322 may be rigidly coupled to the flexible diaphragm 214 and a distal end 323 of the coil spring 322 may be rigidly coupled to the body 202. In other implementations, the coil spring 322 may be unattached to the flexible diaphragm 214, the body 202, or both. For example, in some instances, an end of the coil spring 322 may abut, either directly or indirectly, the flexible diaphragm 214.

The coil spring 322 provides a biasing force that biases the flexible diaphragm 214 and the inner cutter 208 in the direction of the arrow 230, such as when the flexible diaphragm 214 is displaced in the direction of the arrow 240. The coil spring 322 operates to return the flexible diaphragm 214 and the inner cutter 208 back to an initial position in the direction of the arrow 230 once pressure within the diaphragm chamber 312 has been reduced to a selected level or removed altogether. Although the coil spring 322 is provided as an example biasing element operable to provide a biasing force to the flexible diaphragm 214, the scope of the disclosure is not so limited. Rather, any type of spring, such as a Belleville washer, a torsion spring, an extension spring, or any other type of spring may be used.

The inner cutter 208 may be rigidly coupled to flexible diaphragm 214 such that, when flexible diaphragm 214 is displaced by a pressure within diaphragm chamber 312 that is sufficient to overcome the biasing force of coil spring 322, the inner cutter 208 is actuated. When an oscillatory pressure is supplied to the diaphragm chamber 312, the inner cutter 208 actuates in a reciprocal manner at a desired cutting rate. As the inner cutter 208 is actuated, the distal end 209 of inner cutter 208 moves distally in the direction of arrow 240 from a position proximal to the proximal end 213 of aspiration port 210 to a position distal of the distal end 215 of the aspiration port 210. The vitrectomy probe 200d may be configured so that, when the pressure within diaphragm chamber 312 is not sufficient to overcome the biasing force of the coil spring 322, the inner cutter 208 is actuated in the direction of arrow 230 and when the pressure within the diaphragm chamber 312 is sufficient to overcome the biasing force of the coil spring 322, the inner cutter 208 is actuated in the direction of arrow 240.

A power stream port 216, a control jet port 218, and a vent port 220 are each fluidically coupled to a pressure control device 112. The power stream port 216 and the control jet port 218 are also fluidically coupled to the fluidic amplifier 700. The pressure control device 112 supplies fluid to the vitrectomy probe 200d for actuation of the inner cutter 208. The processor 110 and the pressure control device 112 regulate parameters, for example, pressure level and cycle frequency, of the power stream and control jet.

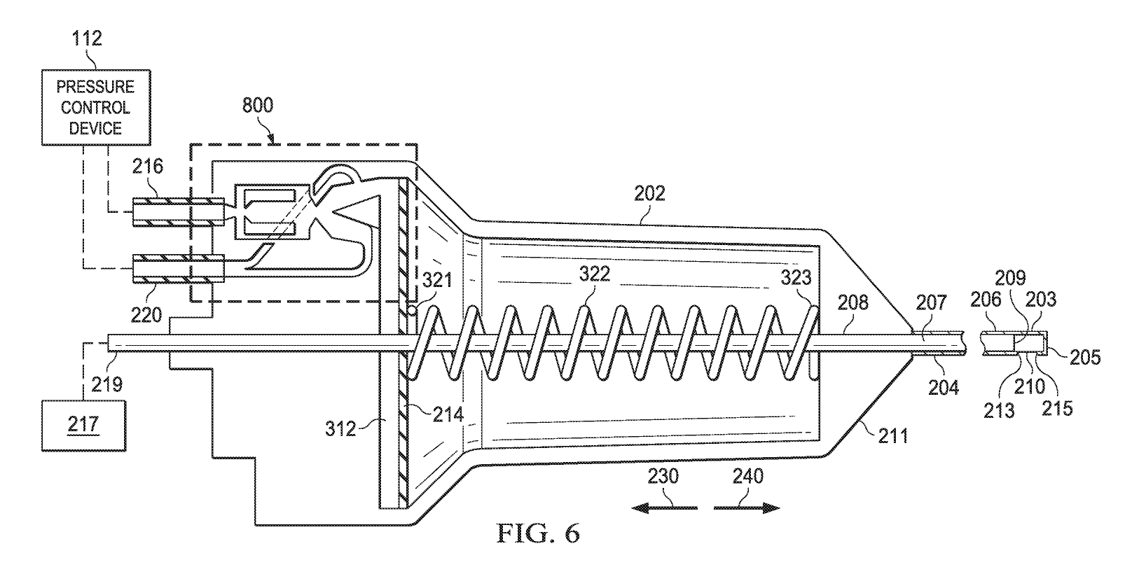

FIG. 6 is a schematic representation of another example vitrectomy probe 200e. The vitrectomy probe 200e is similar to the vitrectomy probe 200d shown in FIG. 5. In particular, the body 202, the cutter 203, the outer cutter 206, the passage 207, the inner cutter 208, the aspiration port 210, the diaphragm chamber 312, and the flexible diaphragm 214 all function and interact in the same way as described above in reference to FIG. 5.

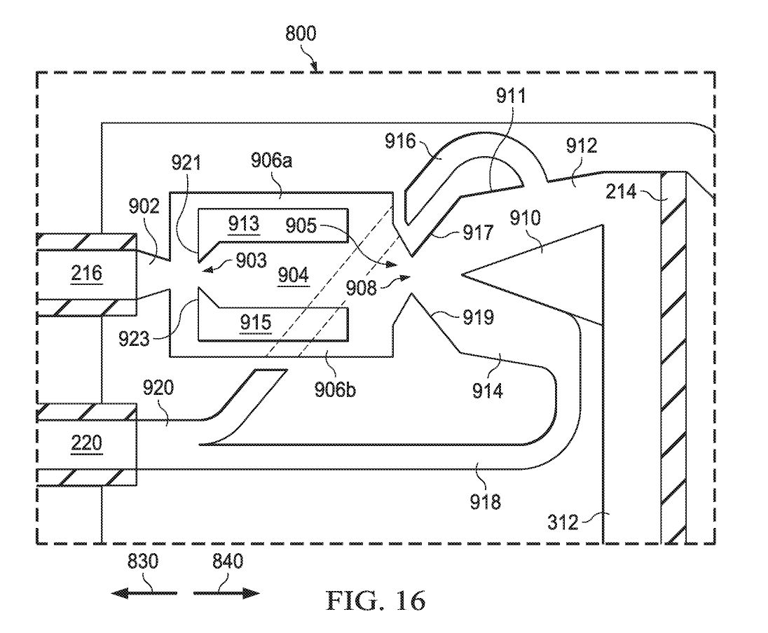

The vitrectomy probe 200e also includes a fluidic oscillator 800, a power stream port 216, and a vent port 220. As shown in FIG. 6, the fluidic oscillator 800 is a general representation of an example fluidic oscillator that is further described in reference to FIG. 16. FIG. 6 illustrates a vitrectomy probe 200e that does not require a control jet port to function. The fluidic oscillator 800 included within the vitrectomy probe 200e is configured to operate without the need for a control jet port. Otherwise the power stream port 216 and the vent port 220 operate in the same way as already described above in reference to FIG. 5. In FIG. 6, the power stream port 216 and the vent port 220 are fluidically coupled to the fluidic oscillator 800.

FIG. 7 is a schematic representation of a fluidic amplifier of a type included in the vitrectomy probe 200a. The fluidic amplifier 400a functions to actuate the inner cutter 208 of the vitrectomy probe 200a. The fluidic amplifier 400a has a power stream inlet 402, a first control jet inlet 404, a second control jet inlet 406 and an interaction region 408a. The power stream inlet 402 is fluidically coupled to the power stream port 216. The first control jet inlet 404 is fluidically coupled to the control jet port 218. The second control jet inlet 406 is fluidically coupled to the control jet port 222. The power stream inlet 402, the first control jet inlet 404, and the second control jet inlet 406 feed into or connect to the proximal end 407 of interaction region 408a. The fluidic amplifier 400a also includes a splitter 410, a distal pressure outlet 412, and a proximal pressure outlet 414. The splitter 410 is located at the distal end 409 of the interaction region 408a and redirects flow from the power stream inlet 402, the first control jet inlet 404, and the second control jet inlet 406. The splitter 410 is aligned with the power stream inlet 402 so that direct flow from the power stream inlet 402 is substantially equally split between the distal pressure outlet 412 and the proximal pressure outlet 414. As shown, the distal pressure outlet 412 is fluidically coupled to the distal diaphragm chamber 212b, and the proximal pressure outlet 414 is fluidically coupled to the proximal diaphragm chamber 212a. The term “substantially” in the context of “substantially equal” means that the described items, e.g., fluid flows, are essentially the same but may experience slight variations. In the context of fluid flows, the slight variations may be the result of continuous fluctuations during operation of fluidic amplifiers or fluidic oscillators described herein, slight variations in the configurations described herein (e.g., a slight variances in the position of the splitter relative to the outlets), or other aspects that may cause slight variances so as to cause the flows to vary slightly from being equal.

The fluidic amplifier 400a also has a distal vent line 416, a proximal vent line 418, and a main vent line 420. The distal vent line 416 is fluidically coupled to the distal pressure outlet 412. The distal vent line 416 is positioned so that, when pressure increases in the distal diaphragm chamber 212b, excess pressure can be vented through the distal vent line 416 so as to prevent backflow through the interaction region 408a. The proximal vent line 418 is fluidically coupled to the proximal pressure outlet 414. The proximal vent line 418 is positioned so that, when pressure increases in the proximal diaphragm chamber 212a, excess pressure can be vented through the proximal vent line 418 so as to prevent backflow through the interaction region 408a. Both the distal vent line 416 and the proximal vent line 418 are fluidically coupled to the main vent line 420. The main vent line 420 is fluidically coupled to the vent port 220. The fluid that is vented through vent port 220 may then be exhausted at a location remote from the vitrectomy probe 200, for example, at the surgical console. In other instances, fluid exhausted through the vent port 220 may be exhausted from the vitrectomy probe 200a directly to the environment.

When the control jets supplied by the first control jet inlet 404 and the second control jet inlet 406 are inactive, the power stream from the power stream inlet 402 freely moves through the interaction region 408a. The splitter 410 divides the power stream from the power stream inlet 402 into two separate flows. The separate flows are directed into the distal pressure outlet 412 and the proximal pressure outlet 414, respectively. In this configuration, the pressure in both the proximal diaphragm chamber 212a and the distal diaphragm chamber 212b are essentially equal. When a first control jet supplied to the first control jet inlet 404 is active and a second control jet supplied to the second control jet inlet 406 is inactive, the first control jet from the first control jet inlet 404 interacts with the power stream from the power stream inlet 402 within the interaction region 408a. The resulting flow passes over the splitter 410 in such a way that the majority of the flow is redirected to the distal pressure outlet 412. The first control jet from the first control jet inlet 404 is fluidically amplified by the power stream from power stream inlet 402. In this configuration, the pressure in the distal diaphragm chamber 212b is greater than the pressure in the proximal diaphragm chamber 212a. This pressure differential causes the flexible diaphragm 214 to become displaced in the direction of arrow 430, thereby actuating the inner cutter 208 in the direction of arrow 430.

When the second control jet supplied to the second control jet inlet 406 is active and the first control jet supplied to the first control jet inlet 404 is inactive, the second control jet from the second control jet inlet 406 interacts with the power stream from the power stream inlet 402 within the interaction region 408a. The resulting flow passes over the splitter 410 in such a way that the majority of the flow is redirected to the proximal pressure outlet 414. The second control jet from the second control jet inlet 406 is fluidically amplified by the power stream from the power stream inlet 402. In this configuration, the pressure in the proximal diaphragm chamber 212a is greater than the pressure in the distal diaphragm chamber 212b. This pressure differential causes the flexible diaphragm 214 to become displaced in the direction of the arrow 440, thereby actuating the inner cutter 208 in the direction of the arrow 440. The first and second control jets from the first and second control jet inlets 404 and 406 are alternatingly applied or cycled such that the flexible diaphragm 214 and inner cutter 208 continue to actuate in a reciprocal manner.

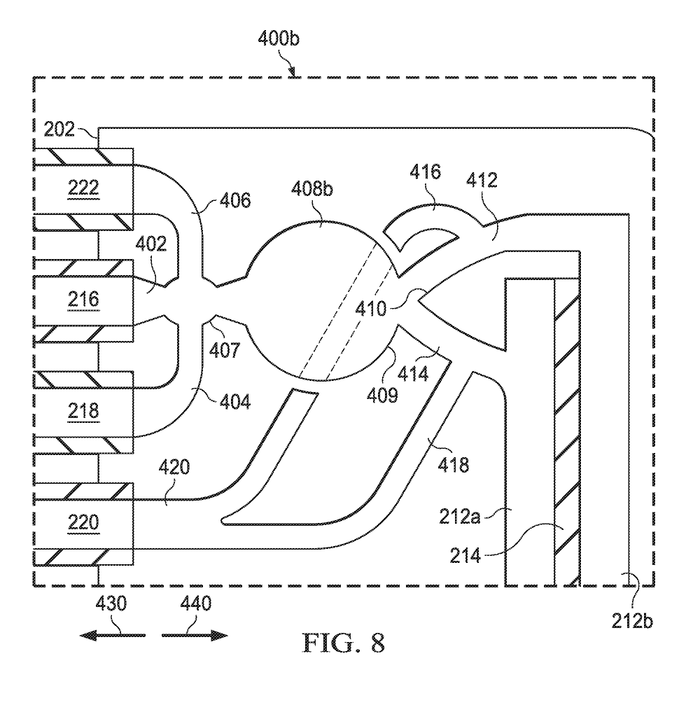

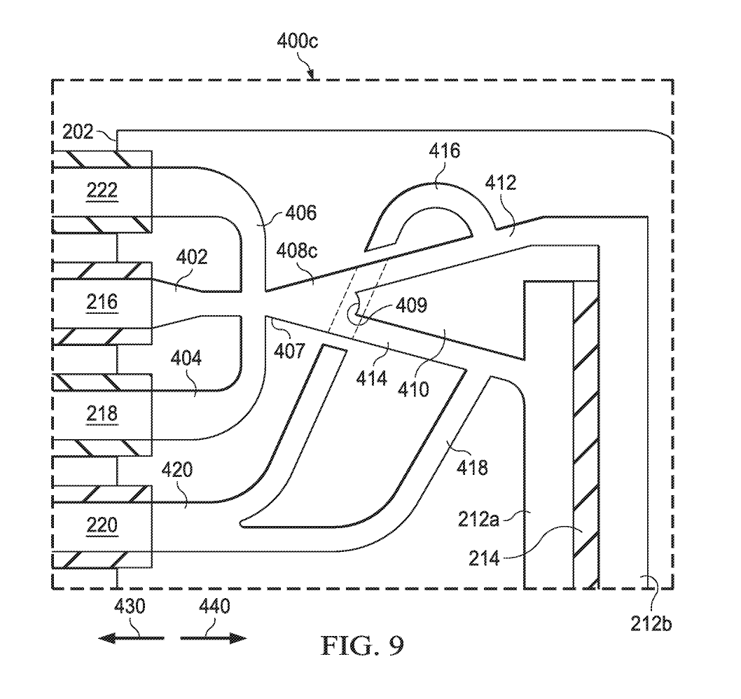

FIGS. 8 and 9 are schematic representations of additional example fluidic amplifiers 400b and 400c, respectively. The fluidic amplifiers 400b and 400c may be used to actuate the inner cutter 208 of the vitrectomy probe 200a in place of the fluidic amplifier 400a. FIGS. 8 and 9 illustrate how the geometry of the interaction region 408 could be modified without changing the functionality of the fluidic amplifier 400 or the vitrectomy probe 200. For example, the interaction region 408b of the fluidic amplifier 400b includes two distinct regions. Flow from the power stream inlet 402, the first control jet inlet 404, and the second control jet inlet 406 (i.e., the power stream, the first control jet, and the second control jet, respectively) enters a proximal portion 407 of the interaction region 408b. Flow circulates within this proximal portion 407 of the interaction region 408b before passing through a distal portion 409 of the interaction region 408b. Otherwise, fluidic amplifier 400b functions similarly to the fluidic amplifier 400a, as described above.

The fluidic amplifier 400c includes the interaction region 408c that is aerodynamically shaped in order to take advantage of the Coanda effect, also referred to as “wall effects.” Flow from the power stream inlet 402, the first control jet inlet 404, and the second control jet inlet 406 enters the interaction region 408c. Both the shape of the walls of the interaction region 408c and the interaction between the power stream from the power stream inlet 402 and either of the control jets from the first control jet inlet 404 or the second control jet inlet 406 redirect flow in the interaction region 408c. Particularly, the first control jet interacts with the power stream to direct the combined flow into the distal pressure outlet 412 to increase pressure within the distal diaphragm chamber 212b, and the second control jet interacts with the power stream to direct the combined flow into the proximal pressure outlet 414 to increase pressure within the proximal diaphragm chamber 212a. Otherwise, the fluidic amplifier 400c functions similarly to the fluidic amplifier 400a and 400b, as described above.

FIG. 10 is a schematic representation of a fluidic amplifier of a type included in the vitrectomy probe 200b. The fluidic amplifier 500a functions to actuate the inner cutter 208 of the vitrectomy probe 200b. The fluidic amplifier 500a is similar to the fluidic amplifier 400a as shown in FIG. 7. In particular, the fluidic amplifier 500a includes a power stream inlet 402, a control jet inlet 404, a splitter 410, a distal pressure outlet 412, a proximal pressure outlet 414, a distal vent line 416, a proximal vent line 418, and a main vent line 420 that all function and interact in the same way as described above with reference to FIG. 7. However, the fluidic amplifier 500a omits the second control jet inlet 406, shown in FIG. 7, and includes a modified interaction region 408d. The power stream inlet 402 is fluidically coupled to the power stream port 216. The control jet inlet 404 is fluidically coupled to the control jet port 218. The power stream inlet 402 and the control jet inlet 404 feed into a proximal end 407 of the interaction region 408d. The splitter 410 is located at a distal end 409 of the interaction region 408d and redirects flow from the power stream inlet 402 and the control jet inlet 404. The splitter 410 is offset from the power stream inlet 402 so that direct flow from the power stream inlet 402, i.e., the power stream, flows into the proximal pressure outlet 414. As shown, the distal pressure outlet 412 is fluidically coupled to the distal diaphragm chamber 212b, and the proximal pressure outlet 414 is fluidically coupled to the proximal diaphragm chamber 212a.

When the control jet supplied by the control jet inlet 404 is inactive, the power stream from the power stream inlet 402 moves undeflected through the interaction region 408d. The splitter 410 redirects the power stream from the power stream inlet 402 into the proximal pressure outlet 414. As a result, the pressure in the proximal diaphragm chamber 212a becomes greater than the pressure in the distal diaphragm chamber 212b. This pressure differential causes the flexible diaphragm 214 to become displaced in the direction of the arrow 540, thereby actuating the inner cutter 208 in the direction of the arrow 540. When the control jet supplied to the control jet inlet 404 is active, the control jet from control jet inlet 404 interacts with the power stream from the power stream inlet 402 within the interaction region 408d. The resulting flow passes over the splitter 410 in such a way that the majority of the flow is redirected to the distal pressure outlet 412. The control jet from control jet inlet 404 is fluidically amplified by the power stream from the power stream inlet 402. As a result, the pressure in the distal diaphragm chamber 212b becomes greater than the pressure in the proximal diaphragm chamber 212a. This pressure differential causes the flexible diaphragm 214 to become displaced in the direction of the arrow 530, thereby actuating the inner cutter 208 in the direction of the arrow 530. Cycling the control jet on and off results in reciprocal actuation of the flexible diaphragm 214 and the inner cutter 208.

FIG. 11 is a schematic representation of another fluidic amplifier of a type included in the vitrectomy probe 200b. The fluidic amplifier 500b functions to actuate the inner cutter 208 of vitrectomy probe 200b. The fluidic amplifier 500b is similar to the fluidic amplifier 500a shown in FIG. 10. In particular, the fluidic amplifier 500b includes a power stream inlet 402, a control jet inlet 404, a distal vent line 416, a proximal vent line 418, and a main vent line 420 that all function and interact in the same way as described above with reference to FIG. 10. However, the fluidic amplifier 500b includes an interaction region 508. The power stream inlet 402 is fluidically coupled to the power stream port 216. The control jet inlet 404 is fluidically coupled to the control jet port 218. The power stream inlet 402 and the control jet inlet 404 feed into a proximal end 507 of the interaction region 508. As shown, the fluidic amplifier 500b also includes a splitter 510, a distal pressure outlet 512, and a proximal pressure outlet 514. The splitter 510 is located at a distal end 509 of interaction region 508 and redirects flow from the power stream inlet 402 and the control jet inlet 404. A lateral sidewall 511 of the interaction region 508 is aerodynamically shaped in order to take advantage of the Coanda effect. The splitter 510 is partially offset from the power stream inlet 402. As a result of the offset of the splitter 510 relative to the power stream inlet 402 and the shape of the lateral sidewall 511 of the interaction region 508, the power stream from the power stream inlet 402, undeflected by a control jet, flows essentially entirely into distal pressure outlet 512. The distal pressure outlet 512 is fluidically coupled to the distal diaphragm chamber 212b, and the proximal pressure outlet 514 is fluidically coupled to the proximal diaphragm chamber 212a.

The distal vent line 416 is fluidically coupled to the distal pressure outlet 512. The distal vent line 416 is positioned so that, when the pressure increases in the distal chamber 212b, excess pressure is vented, preventing backflow through the interaction region 508. The proximal vent line 418 is fluidically coupled to the proximal pressure outlet 514. The proximal vent line 418 is positioned so that, when the pressure increases in the proximal chamber 212a, excess pressure is vented, preventing backflow through the interaction region 508.

When the control jet supplied by the control jet inlet 404 is inactive, the flow, i.e., the power stream, from power stream inlet 402 moves undeflected through interaction region 508 while remaining attached to the lateral sidewall 511 of the interaction region 508 due to the Coanda effect. The splitter 510 is positioned to direct the flow from power stream inlet 402 into distal pressure outlet 512. As a result, the pressure in the distal diaphragm chamber 212b increases above the pressure in the proximal diaphragm chamber 212a. This pressure differential causes the flexible diaphragm 214 to become displaced in the direction of the arrow 530, thereby actuating the inner cutter 208 in the direction of the arrow 530. When the control jet supplied to control jet inlet 404 is active, the control jet from the control jet inlet 404 interacts with the power stream from the power stream inlet 402 within the interaction region 508. This interaction interferes with the Coanda effect, and the power stream from the power stream inlet 402 detaches from the lateral sidewall 511 of interaction region 508. The resulting flow passes over the splitter 510 in such a way that the majority of the flow is redirected to the proximal pressure outlet 514. The control jet from control jet inlet 404 is fluidically amplified by the power stream from the power stream inlet 402. As a result, the pressure in the proximal diaphragm chamber 212a increases above the pressure in the distal diaphragm chamber 212b. This pressure differential causes the flexible diaphragm 214 to become displaced in the direction of the arrow 540, thereby actuating the inner cutter 208 in the direction of the arrow 540. Cycling the control jet on and off results in reciprocal actuation of the flexible diaphragm 214 and the inner cutter 208.

FIG. 12 is a schematic representation of an example fluidic oscillator of a type included in the vitrectomy probe 200c. The fluidic oscillator 600 functions to actuate the inner cutter 208 of the vitrectomy probe 200c. The fluidic oscillator 600 includes a power stream inlet 602, feedback channels 606a and 606b, and an interaction region 604. The power stream inlet 602 is fluidically coupled to the power stream port 216. The power stream inlet 602 and the feedback channels 606a and 606b feed into the proximal end 603 of the interaction region 604. The fluidic oscillator 600 also includes a nozzle 608, a splitter 610, a distal pressure outlet 612, and a proximal pressure outlet 614. The nozzle 608 is located at the distal end 605 of the interaction region 604. The splitter 610 is located distal to the nozzle 608 at the distal end 611 of the fluidic oscillator 600 and redirects the power stream from the power stream inlet 602 after the power stream passes through the interaction region 604 and the nozzle 608.

The fluidic oscillator 600 also includes a first wall 613, a second wall 615, a first tapered sidewall portion 617 and a second tapered sidewall portion 619. The first wall 613 includes a tapered wall portion 621, and the second wall 615 includes a tapered wall portion 623. The tapered wall portions 621 and 623 taper to respective edges that define the proximal end 603 of the interaction region 604. The first and second tapered sidewall portions 617 and 619 taper to respective edges that define the nozzle 608.

The power stream from the power stream inlet 602 initially passes undeflected through the interaction region 604. However, the structure of the fluidic oscillator 600, including the first wall 613 and the second wall 615 of the interaction region 604 and the first pointed sidewall portion 617 and second pointed sidewall portion 619 that form the nozzle 608, introduce instabilities into the power stream from power stream inlet 602. For example, as the power stream from the power stream inlet 602 passes through a region between a tapered wall portion 621 of the first wall 613 and a tapered wall portion 623 of the second wall 615 of the interaction region 604, the power stream may be redirected toward the first wall 613 by the tapered wall portion 623 of the second wall 615. As the redirected power stream continues through the interaction region 604, the redirected power stream may collide with the first wall 613 and then continue toward the nozzle 608. The power stream may then collide with the first tapered sidewall portion 617 that forms the nozzle 608. In this situation, the nozzle 608 redirects some of the flow through the nozzle 608 toward the splitter 610 and downward toward the proximal pressure outlet 614. However, some of the flow may backup after colliding with the first tapered sidewall portion 617 of nozzle 608. The backed up portion of the flow may cause flow to travel in the direction of arrow 630 through feedback channel 606a. The flow from feedback channel 606a along with the tapered wall portion 621 of the first wall 613 of the interaction region 604 may then redirect the flow from power stream inlet 602 toward the second wall 615 of the interaction region 604.

As the flow continues through the interaction region 604, the redirected flow may collide with the second wall 615 and then continue toward the nozzle 608. The flow may then collide with the second tapered sidewall portion 619 that forms the nozzle 608. In this situation, the nozzle 608 redirects some of the flow through the nozzle 608 toward the splitter 610 and upward toward the distal pressure outlet 612. However, some of the flow may backup after colliding with the second tapered sidewall portion 619 of the nozzle 608. The flow that has backed up may cause flow to travel in the direction of arrow 630 through feedback channel 606b. The flow from the feedback channel 606b along with the tapered wall portion 623 of the second wall 615 of the interaction region 604 may then redirect the flow from the power stream inlet 602 toward the first wall 613 of the interaction region 604. In this way, the flow from the power stream inlet 602 will continue to oscillate. This oscillation will cause the flow that exits from the nozzle 608 to pass over the splitter 610 in such a way that the majority of the flow is redirected into either the distal pressure outlet 612 or the proximal pressure outlet 614, depending on how the flow exits from the nozzle 608. As shown, the distal pressure outlet 612 is fluidically coupled to the distal diaphragm chamber 212b and the proximal pressure outlet 614 is fluidically coupled to the proximal diaphragm chamber 212a. The fluidic oscillator 600 also has a distal vent line 616, a proximal vent line 618, and a main vent line 620 that function and interact in the same way as described above in reference to FIG. 2 through FIG. 11.

When the power stream exits the nozzle 608 in such a way that redirects the flow toward the splitter 610 and the distal pressure outlet 612, the pressure in the distal diaphragm chamber 212b becomes greater than the pressure in the proximal diaphragm chamber 212a. This pressure differential causes the flexible diaphragm 214 to become displaced in the direction of the arrow 630, thereby actuating the inner cutter 208 in the direction of the arrow 630. When the power stream exits the nozzle 608 in such a way that redirects the flow toward the splitter 610 and the proximal pressure outlet 614, the pressure in the proximal diaphragm chamber 212a becomes greater than the pressure in the distal diaphragm chamber 212b. This pressure differential causes the flexible diaphragm 214 to become displaced in the direction of the arrow 640, thereby actuating the inner cutter 208 in the direction of the arrow 640. Continuously supplying the power stream results in reciprocal actuation of the flexible diaphragm 214 and the inner cutter 208.

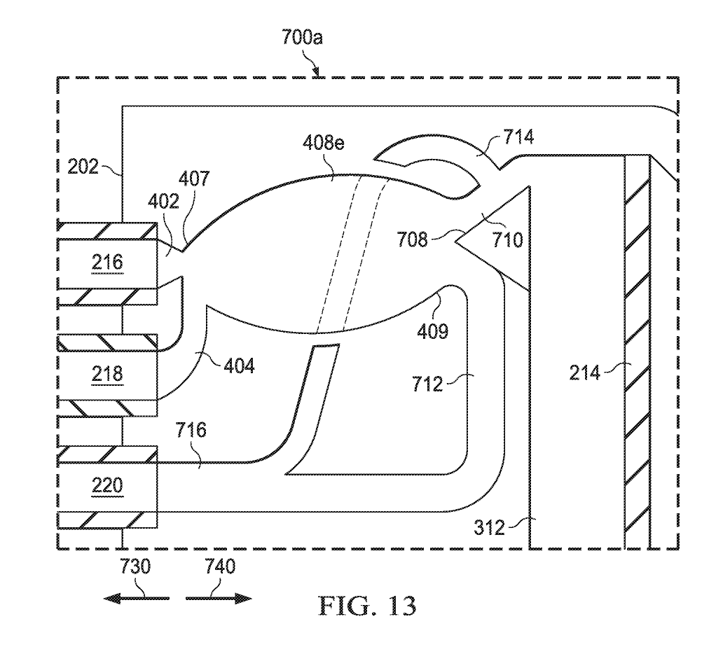

FIG. 13 is a schematic representation of a fluidic amplifier of a type included in the vitrectomy probe 200d shown in FIG. 5. The fluidic amplifier 700a functions to actuate the inner cutter 208 of the vitrectomy probe 200d. The fluidic amplifier 700a is similar to the fluidic amplifier 500a shown in FIG. 10. In particular, the fluidic amplifier 700a includes a power stream inlet 402, a control jet inlet 404, and an interaction region 408e. The power stream inlet 402 is fluidically coupled to the power stream port 216. The control jet inlet 404 is fluidically coupled to the control jet port 218. The power stream inlet 402 and the control jet inlet 404 feed into the proximal end 407 of the interaction region 408e. As shown, the fluidic amplifier 700a also has a splitter 708, an active pressure outlet 710, and an exhaust pressure outlet 712. The splitter 708 is located at the distal end 409 of the interaction region 408e and redirects flow from the power stream inlet 402 and the control jet inlet 404. The active pressure outlet 710 conducts fluid that is used to actuate the flexible diaphragm 214. The splitter 708 is offset from the power stream inlet 402 so that direct flow from power stream inlet 402, i.e., the power stream, flows into the exhaust pressure outlet 712. As shown, the active pressure outlet 710 is fluidically coupled to the diaphragm chamber 312.

The fluidic amplifier 700a also includes an active vent line 714 and a main vent line 716. The active vent line 714 is a vent line associated with active pressure outlet 710 that is used to conduct fluid to actuate the flexible diaphragm 214. The active vent line 714 is fluidically coupled to the active pressure outlet 710. The active vent line 714 is positioned so that, when pressure increases in the diaphragm chamber 312, excess pressure is vented through the active vent line 714 so as to prevent backflow through the interaction region 408e. As shown, the active vent line 714 and the exhaust pressure outlet 712 are fluidically coupled to the main vent line 716. The main vent line 716 is fluidically coupled to the vent port 220. The fluid that is vented through vent port 220 is then exhausted at a location remote from the vitrectomy probe 200d, for example, at the surgical console. In other instances, fluid exhausted through the vent port 220 may be exhausted from the vitrectomy probe 200d directly to the environment.

When the control jet supplied by the control jet inlet 404 is inactive, the power stream from the power stream inlet 402 moves undeflected through the interaction region 408e. The splitter 708 is positioned to direct the power stream from the power stream inlet 402 into the exhaust pressure outlet 712. As a result, the pressure in the diaphragm chamber 312 is not sufficient to overcome the biasing force of the coil spring 322 (shown in FIG. 5) and the flexible diaphragm 214 (also shown in FIG. 5) either remains stationary or becomes displaced in the direction of arrow 730 due to the biasing force of the coil spring 322, thereby actuating the inner cutter 208 in the direction of the arrow 730. When the control jet supplied to the control jet inlet 404 is active, the control jet from the control jet inlet 404 interacts with the power stream from the power stream inlet 402 within the interaction region 408e. The resulting flow passes over the splitter 708 in such a way that the majority of the flow is redirected to the active pressure outlet 710. The control jet from the control jet inlet 404 is fluidically amplified by the power stream from the power stream inlet 402. This amplified pressure in the diaphragm chamber 312 is sufficient to overcome the biasing force of the coil spring 322 and causes flexible diaphragm 214 to become displaced in the direction of the arrow 740, thereby actuating the inner cutter 208 in the direction of the arrow 740. Cycling the control jet on and off results in reciprocal actuation of the flexible diaphragm 214 and inner cutter 208 (shown in FIG. 5).

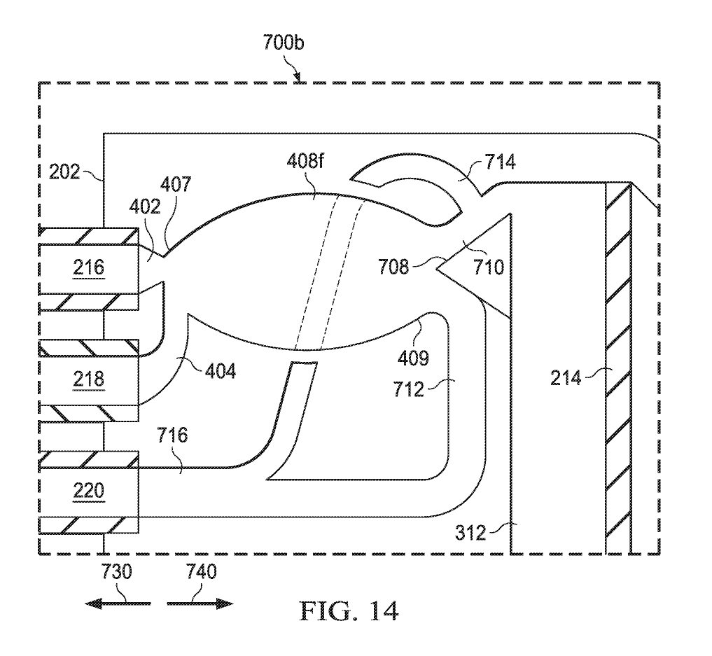

FIG. 14 is a schematic representation of a fluidic amplifier of a type included in the vitrectomy probe 200d, shown in FIG. 5. FIG. 14 shows a variation of the example fluidic amplifier shown in FIG. 13. However, the splitter 708 of the fluidic amplifier 700b is positioned aligned with the power stream port 216. The splitter 708 of the fluidic amplifier 700a of FIG. 13 is offset from the power stream port 216. In other implementations, the position of the splitter relative to the power stream port may be varied to achieve a desired output. The fluidic amplifier 700b functions to actuate the inner cutter 208 of the vitrectomy probe 200d. The fluidic amplifier 700b is similar to the fluidic amplifier 700a as shown in FIG. 13. In particular, the fluidic amplifier 700b includes a power stream inlet 402, a control jet inlet 404, a splitter 708, an active pressure outlet 710, an exhaust pressure outlet 712, an active vent line 714, and a main vent line 716 that all function and interact in the same way as described in reference to FIG. 13. However, the fluidic amplifier 700b includes an interaction region 408f. The power stream inlet 402 is fluidically coupled to the power stream port 216. The control jet inlet 404 is fluidically coupled to the control jet port 218. The power stream inlet 402 and the control jet inlet 404 feed into the proximal end 407 of interaction region 408f The splitter 708 is aligned with the power stream inlet 402 so that direct flow from the power stream inlet 402 is equally split between the active pressure outlet 710 and the exhaust pressure outlet 712.

When the control jet supplied by the control jet inlet 404 is inactive, the power stream from the power stream inlet 402 moves undeflected through the interaction region 408f. The splitter 708 divides the power stream from the power stream inlet 402 into two separate flows. The separate flows are directed into the active pressure outlet 710 and the exhaust pressure outlet 712, respectively. In this configuration, the pressure in the diaphragm chamber 312 is not sufficient to overcome the biasing force of the coil spring 322, and the flexible diaphragm 214 either remains stationary or becomes displaced in the direction of arrow 730 by the biasing force of the coil spring 322, thereby actuating the inner cutter 208 in the direction of arrow 730. When the control jet supplied to the control jet inlet 404 is active, the control jet from the control jet inlet 404 interacts with the power stream from the power stream inlet 402 within interaction region 408f The resulting flow passes over the splitter 708 in such a way that the majority of the flow is redirected to the active pressure outlet 710. The control jet from the control jet inlet 404 is fluidically amplified by the power stream from the power stream inlet 402. This amplified pressure in the diaphragm chamber 312 is sufficient to overcome the biasing force of the coil spring 322 and causes flexible diaphragm 214 to become displaced in the direction of arrow 740, thereby actuating inner cutter 208 in the direction of arrow 740. Cycling the control jet on and off results in reciprocal actuation of the flexible diaphragm 214 and the inner cutter 208.

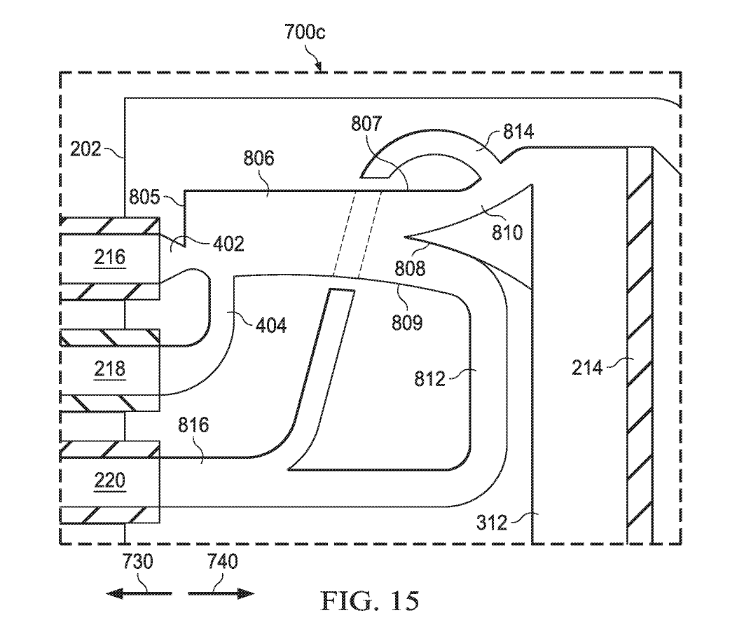

FIG. 15 is a schematic representation of another fluidic amplifier of a type included in the vitrectomy probe 200d, again, shown in FIG. 5. The fluidic amplifier 700c functions to actuate the inner cutter 208 of the vitrectomy probe 200d. The fluidic amplifier 700c is similar to the fluidic amplifier 500b shown in FIG. 11. In particular, the fluidic amplifier 700c includes a power stream inlet 402 and a control jet inlet 404 that function and interact in the same way as described in reference to FIG. 11. However, the fluidic amplifier 700c includes an interaction region 806. The power stream inlet 402 is fluidically coupled to the power stream port 216. The control jet inlet 404 is fluidically coupled to the control jet port 218. The power stream inlet 402 and the control jet inlet 404 feed into the proximal end 805 of the interaction region 806. As shown, the fluidic amplifier 700c also has a splitter 808, an active pressure outlet 810, and an exhaust pressure outlet 812. The splitter 808 is located at the distal end 807 of the interaction region 806 and redirects flow from the power stream inlet 402 and the control jet inlet 404. A lateral sidewall 809 of the interaction region 806 is aerodynamically shaped in order to take advantage of the Coanda effect. The splitter 808 is partially offset from the power stream inlet 402. As a result of the offset of the splitter 808 relative to the power stream inlet 402 and the shape of the lateral sidewall 809 of the interaction region 806, the power stream from the power stream inlet 402 flows essentially entirely into the exhaust pressure outlet 812. As shown in FIG. 15, the active pressure outlet 810 is fluidically coupled to the diaphragm chamber 312.

The fluidic amplifier 700c also includes an active vent line 814 and a main vent line 816. The active vent line 814 is fluidically coupled to the active pressure outlet 810. The active vent line 814 is positioned so that, when pressure increases in the diaphragm chamber 312, excess pressure is vented through the active vent line so as to prevent backflow through the interaction region 806. Both the distal vent line 814 and the exhaust pressure outlet 812 are fluidically coupled to the main vent line 816. The main vent line 816 is fluidically coupled to the vent port 220. The fluid that is vented through the vent port 220 is then exhausted at a location remote from the vitrectomy probe 200d, for example, at the surgical console. In other instances, fluid exhausted through the vent port 220 may be exhausted from the vitrectomy probe 200d directly to the environment.

When the control jet supplied by the control jet inlet 404 is inactive, the power stream from the power stream inlet 402 moves undeflected through interaction region 806 and remains attached to the lateral sidewall 809 of the interaction region 806 due to the Coanda effect. The splitter 808 is positioned to direct the flow from the power stream inlet 402 into the exhaust pressure outlet 812. As a result, the pressure in the diaphragm chamber 312 is not sufficient to overcome the biasing force of the coil spring 322, and the flexible diaphragm 214 either remains stationary or is actuated in the direction of arrow 730 by the biasing force of the actuating spring 322, thereby actuating the inner cutter 208 in the direction of arrow 730. When the control jet supplied to the control jet inlet 404 is active, the control jet from the control jet inlet 404 interacts with the power stream from the power stream inlet 402 within the interaction region 806. This interaction interferes with the Coanda effect, and the power stream from the power stream inlet 402 detaches from the lateral sidewall 809 of the interaction region 806. The resulting flow passes over the splitter 808 in such a way that the majority of the flow is redirected to the active pressure outlet 810. The control jet from the control jet inlet 404 is fluidically amplified by the power stream from the power stream inlet 402. This amplified pressure in the diaphragm chamber 312 is sufficient to overcome the biasing force of the coil spring 322 and causes flexible diaphragm 214 to become displaced in the direction of the arrow 740, thereby actuating inner cutter 208 in the direction of the arrow 740. Cycling the control jet on and off results in reciprocal actuation of the flexible diaphragm 214 and the inner cutter 208.

FIG. 16 is a schematic representation of an example fluidic oscillator of a type included in the vitrectomy probe 200e, shown in FIG. 6. The fluidic oscillator 800 functions to actuate the inner cutter 208 of the vitrectomy probe 200e. The fluidic oscillator 800 is similar to the fluidic oscillator described in reference to FIG. 12. In particular, the fluidic oscillator 800 includes a power stream inlet 902, feedback channels 906a and 906b, an interaction region 904, a nozzle 908, a splitter 910, an active pressure outlet 912, an exhaust pressure outlet 914, a first wall 913, a second wall 915, an active vent line 916, an exhaust vent line 918, and a main vent line 920 that function and interact in the same way as the corresponding components of the fluidic oscillator 600 described above in reference to FIG. 12. The power stream inlet 902 is fluidically coupled to the power stream port 216. The power stream inlet 902 and feedback channels 906a and 906b feed into the proximal end 903 of the interaction region 904.

When the power stream exits the nozzle 908 in such a way that redirects the flow toward the splitter 910 and the active pressure outlet 912, the pressure in diaphragm chamber 312 is sufficient to overcome the biasing force of the coil spring 322 and causes the flexible diaphragm 214 to become displaced in the direction of arrow 840, thereby actuating the inner cutter 208 in the direction of arrow 840. When the power stream exits the nozzle 608 in such a way that redirects the flow toward the splitter 910 and the exhaust pressure outlet 914, the pressure in diaphragm chamber 312 is not sufficient to overcome the biasing force of the coil spring 322 and the flexible diaphragm 214 either remains stationary or becomes displaced in the direction of arrow 830 by the biasing force of the coil spring 322. Continuously supplying the power stream results in reciprocal actuation of the flexible diaphragm 214 and the inner cutter 208.

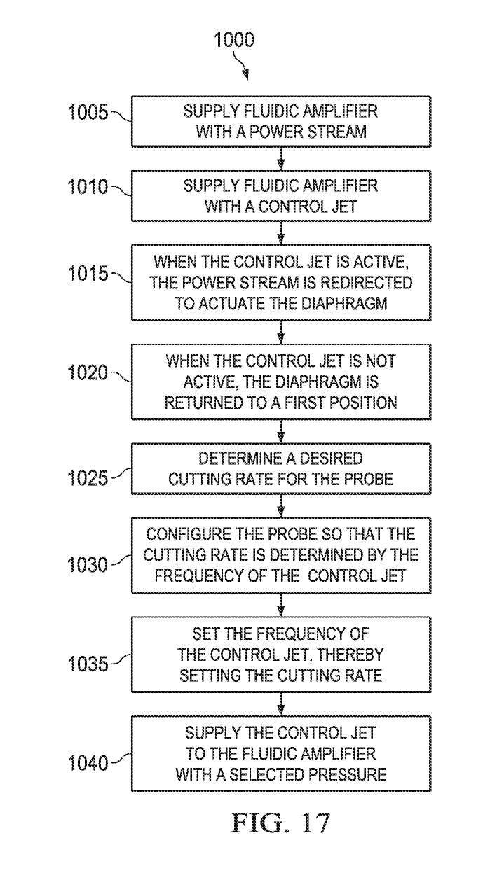

FIG. 17 is a flowchart of an example method 1000 for actuating an inner cutter of a vitrectomy probe. At step 1005, a power stream is supplied to a fluidic amplifier of a vitrectomy probe. The power stream may be supplied via a pressure line or any other appropriate supply structure. The power stream may be supplied at any appropriate or desired pressure level. For example, the power stream pressure level may be based on the characteristics of a diaphragm that is used in an actuating mechanism of the vitrectomy probe. The power stream pressure level may also be set in relation to the level of a control jet pressure, and vice versa. The power stream may result in deflection of a flexible diaphragm, such as flexible diaphragm 214, for example, into either a distal or a proximal position which may depend on, for example, a configuration of the fluidic amplifier, a configuration of the vitrectomy probe, and/or other requirements of a user.

At step 1010, a control jet is supplied to the fluidic amplifier of the vitrectomy probe. In some implementations, the control jet may be a single pulse. In other implementations, the control jet may be a series of pulses. In some instances, these series of pulses may be set to a cyclical pressure profile. The control jet may be supplied by any suitable or desired structure including, for example, by a pressure line, conduit, tubing, or other structure operable to conduct a fluid. The control jet may be supplied at a selected pressure level. The control jet may be cycled between a peak or maximum pressure and a minimum pressure. The minimum pressure of the control jet may be zero pressure or some pressure less than the maximum pressure. For example, in an off or inactive condition, the control jet is being applied at the minimum pressure. In an on or active condition, the control jet is being applied at the maximum or peak pressure. In some instances, the maximum or peak pressure of the control jet may be less than the pressure of the power stream. In some instances, the control jet pressure level may be based on the characteristics of the diaphragm that is used in the actuating mechanism of the vitrectomy probe, such as, for example, mass, rigidity, and/or configuration of the diaphragm. The control jet pressure level may also be set in relation to the level of the power stream pressure.

At step 1015 and step 1020, the fluidic amplifier may be configured such that the control jet and the power stream cooperate to achieve the desired pressure distribution within a diaphragm chamber of the vitrectomy probe that houses the flexible diaphragm, such as, for example, the diaphragm chambers 212 and 312, described above. At step 1015, the control jet is active such that the maximum pressure of the control jet is applied to the power stream. The interaction of the active control jet and the power stream may result in the majority of the power stream being redirected within the fluidic amplifier in such a way that results in actuation (e.g., deflection) of a flexible diaphragm, such as flexible diaphragm 214. The flexible diaphragm may be actuated into a different position than a position than that associated with application of the power stream alone to the fluidic amplifier. The interaction of the active control jet and the power stream also results in an amplified output pressure that is greater than the control jet pressure.

As discussed in the detailed description of FIG. 7 through FIG. 11, the amplified output pressure results in a pressure differential across the flexible diaphragm, which deflects the flexible diaphragm and actuates the inner cutter, such as inner cutter 208. The control jet pressure may be set at any desire level, such as a level sufficient to cause the flexible diaphragm to be actuated. For instance, a pressure of the control jet may selected to produce an amplified output pressure that causes an increase within the diaphragm chamber of, for example, less than 1%, less than 5%, or less than 10% of the power stream pressure. However, other output pressures are within the scope of the disclosure. The pressure of the control jet may be selected so as to cause other pressure differentials within the diaphragm chamber below, above, and between the indicated values are also within the scope of the disclosure. As discussed above in the context of FIG. 13, FIG. 14, and FIG. 15, the amplified output pressure may result in a pressure within the diaphragm chamber that is sufficient to overcome the biasing force of the coil spring, such as coil spring 322, which allows the flexible diaphragm to actuate the inner cutter.

At step 1020, the control jet is not active. Consequently, the control jet is being applied at the minimum pressure. The power stream is unaffected or, wherein the control jet pressure is greater than zero, minimally affected by the inactive control jet. The power stream is redirected within the fluidic amplifier and the flexible diaphragm returns to an initial or first position, which may be either a distal or proximal position as in step 1005.

At step 1025, the user may determine a desired cutting rate for the vitrectomy probe. In determining the desired cutting rate, the user may consider, for example, the stage of the vitrectomy procedure or the location within the eye where vitreous removal is desired. In some instances, the user may determine one or multiple desired cutting rates throughout the vitrectomy procedure. In some implementations, the user may desire a single cutting rate during a vitrectomy, and the vitrectomy probe may be operated to achieve an actuation cycle corresponding to a single cutting rate.

At step 1030, the vitrectomy probe is configured such that the desired cutting rate for the vitrectomy probe is determined by and corresponds to a desired frequency of the control jet pressure that is supplied to the fluidic amplifier. The flexible diaphragm is responsive to the changes in the control jet pressure. The flexible diaphragm may be coupled to the inner cutter such that the inner cutter is actuated at the same frequency as the control jet pressure in response to actuation of the flexible diaphragm. In some instances, the inner cutter may be rigidly coupled to the flexible diaphragm. As such, the frequency of application of the control jet pressure to the power stream corresponds to and determines the cutting rate of the inner cutter.

At step 1035, in order to achieve the desired cutting rate that was determined at step 1025, the frequency of application of the control jet pressure to the power stream may be set at the desired cutting rate. As a result, the vitrectomy probe will operate at the desired cutting rate as the inner cutter 208 is actuated at the desired frequency.

At step 1040, a pressure of the control jet supplied to the power stream may be selected so as to ensure that the flexible diaphragm is in a desired position at the conclusion of a vitrectomy procedure or when the vitrectomy probe is deactivated, such as, for example, when the user causes the vitrectomy probe to cease operation of the cutter. This pressure to ensure the desired positioning of the diaphragm may be referred to as a final pressure profile. For example, the user may desire that the aspiration port be open before removing the vitrectomy probe from the eye. Ensuring that the aspiration port be open may prevent damage to the eye caused by unsevered vitreous strands held by the cutter and still attached to the eye. To ensure an open aspiration port such as by retraction of the inner cutter, the control jet is either applied or not applied to the power stream depending on, for example, the configuration of the fluidic amplifier, the configuration of the vitrectomy probe, and/or on the requirements of the user. After supplying the final pressure profile, the vitrectomy procedure may be concluded, and the vitrectomy probe may be removed from the eye.

Although method 1000 illustrates an example process for actuating an inner cutter of a vitrectomy probe, other methods for actuating the inner cutter may include fewer, additional, and or a different arrangement of operations. For example, a method may omit one or more of the described steps, such as, for example, steps 1025, 1030, 1035, or 1040. Still further, the arrangement of steps illustrated in FIG. 17 may be varied from that described above and shown in FIG. 17.

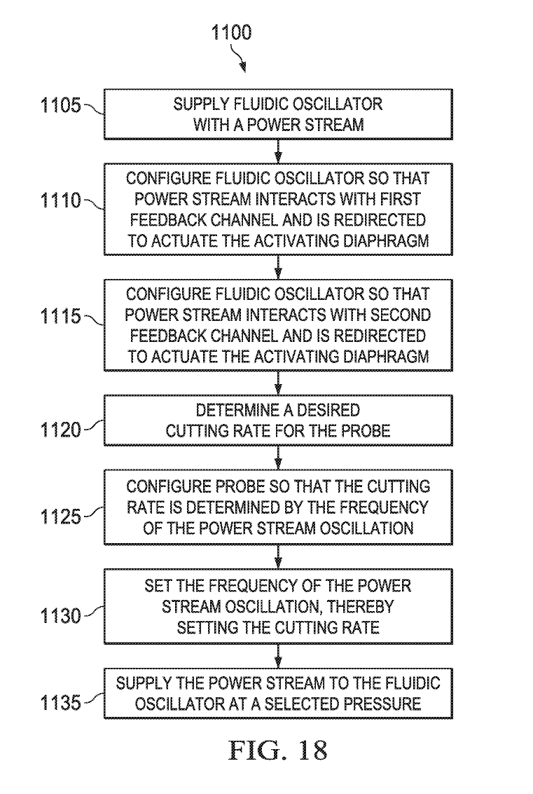

FIG. 18 is a flowchart of an example method 1100 for actuating an inner cutter of a vitrectomy probe. At step 1105, a power stream may be supplied to a fluidic oscillator of a vitrectomy probe. The power stream may be supplied via a pressure line, conduit, tubing, or other structure operable to conduct a fluid. The power stream may be supplied at a selected pressure level. The power stream pressure level may be based on, for example, the characteristics of the diaphragm that is used in the actuating mechanism of the vitrectomy probe. The power stream may result in actuation (e.g., deflection) of the flexible diaphragm into either a distal or proximal position. Actuation of the flexible diaphragm into either the distal or proximal position may be based on a configuration of the fluidic oscillator, a configuration of the vitrectomy probe, and/or on other requirements of the user.

At steps 1110 and 1115, the fluidic oscillator may be configured such that the power stream interacts with feedback channels within the fluidic oscillator to achieve the desired pressure distribution within a diaphragm chamber that houses the flexible diaphragm, such as diaphragm chamber 212 or the diaphragm chamber 312. At step 1110, a first feedback channel is active. A first feedback pressure resulting from the first feedback channel may be at a peak. As a result of the first feedback pressures, the flexible diaphragm may be actuated into a different position than an initial position. The initial position of the flexible diaphragm may correspond to the position of the flexible diaphragm that results from application of the power stream unaffected by feedback pressure. The interaction of the first feedback channel and the power stream may result in the majority of the power stream being redirected within the fluidic oscillator in such a way that the flexible diaphragm 214 is actuated (e.g., deflected). As discussed in the detailed description associated with FIG. 12, the power stream causes a pressure differential across the flexible diaphragm, which actuates the inner cutter. A pressure of the power stream may be selected so as to cause the flexible diaphragm to be actuated. For instance, the power stream pressure may be selected so as to result in a pressure differential within the diaphragm chamber of, for example, less than 1%, less than 5%, or less than 10% of the power stream pressure. Again, though, other output pressures are within the scope of the disclosure. The pressure of the power stream may be selected so as to cause other pressure differentials within the diaphragm chamber below, above, and between the indicated values are also within the scope of the disclosure. As discussed in the detailed description of FIG. 16, the power stream may result in a pressure within the diaphragm chamber that is sufficient to overcome the biasing force of the coil spring, which allows the flexible diaphragm to actuate the inner cutter.

At step 1115, a second feedback channel is active. A second feedback pressure resulting from the second feedback channel may be at a peak. In response to the second feedback pressure, the flexible diaphragm may be actuated into a position different than that associated with the first feedback pressure and different from that resulting from the power stream unaffected by the first or second feedback pressures. The power stream is redirected within the fluidic oscillator due to the second feedback pressure, causing the flexible diaphragm to be actuated in a direction opposite to that caused by the first feedback pressure. As the power stream continues to be supplied to the fluidic oscillator, the power stream will continue to interact with the internal structure of the fluidic oscillator, e.g., the feedback channels, creating the first and second feedback pressures and causing the power stream to oscillate in response thereto. As long as the power stream is supplied to the fluidic oscillator, the power stream will continue to oscillate and actuate diaphragm in a reciprocating manner.

At step 1120, the user may determine a desired cutting rate for the vitrectomy probe. In determining the desired cutting rate, the user may consider, for example, the stage of the vitrectomy procedure or the location within the eye where vitreous removal is desired. In some instances, the user may determine one or multiple desired cutting rates throughout the vitrectomy surgery. The user may also desire a single cutting rate during a vitrectomy, and the vitrectomy probe may be operated to achieve an actuation cycle corresponding to a single cutting rate.

At step 1125, the vitrectomy probe is configured such that the desired cutting rate for the vitrectomy probe is determined by and corresponds to a desired frequency of the power stream oscillation within the fluidic oscillator. The flexible diaphragm is responsive to the changes in the power stream. The flexible diaphragm may be coupled to the inner cutter such that the inner cutter is actuated at the same frequency as the power stream oscillation in response to the flexible diaphragm. In some instances, the inner cutter may be rigidly coupled to the flexible diaphragm. As such, the frequency of the power stream oscillation corresponds to and determines the cutting rate of the inner cutter.

At step 1130, in order to achieve the desired cutting rate that was determined at step 1120, the frequency of the power stream oscillation may be set at the desired cutting rate. Generally, with the use of a fluidic oscillator, the fluidic oscillator would be designed to operate at a desired, fixed frequency. With the use of a fluidic amplifier, the frequency of operation may be adjusted based on the frequency at which the control jet is operated. As a result, the vitrectomy probe will operate at the desired cutting rate as the inner cutter 208 is actuated at the desired frequency.

At step 1135, a pressure of the power stream supplied to the fluidic oscillator may be selected in order to ensure that the flexible diaphragm is in a desired position at the conclusion of a vitrectomy procedure or when the vitrectomy probe is deactivated, such as, for example, when the user causes the vitrectomy probe to cease operation of the cutter. This pressure to ensure the desired positioning of the diaphragm may be referred to as a final pressure profile. For example, the user may require that the aspiration port be open before removing the vitrectomy probe from the eye. As explained above, ensuring that the aspiration port is in an open condition may prevent injury to the eye. To ensure an open aspiration port, such as by retraction of the inner cutter, the power stream is prevented from being supplied to the fluidic oscillator at the appropriate time based on, for example, the configuration of the fluidic oscillator, the configuration of the vitrectomy probe, and/or on the requirements of the user. After supplying the final pressure profile, the vitrectomy procedure may be concluded, and the vitrectomy probe may be removed from the eye.

Although method 1100 illustrates an example process for actuating an inner cutter of a vitrectomy probe, other methods for actuating the inner cutter may include fewer, additional, and or a different arrangement of operations. For example, a method may omit one or more of the described steps, such as, for example, steps 1125, 1130, or 1135. Still further, the arrangement of steps illustrated in FIG. 18 may be varied from that described above and shown in FIG. 18.

The above disclosed subject matter is to be considered illustrative, and not restrictive, and the appended claims are intended to cover all such modifications, enhancements, and other embodiments which fall within the true spirit and scope of the present disclosure. For example, although the above systems and methods are discussed in the context of actuating a vitrectomy probe, a similar system may be used to actuate other surgical instruments that employ fluidic actuators. Thus, to the maximum extent allowed by law, the scope of the present disclosure is to be determined by the broadest permissible interpretation of the following claims and their equivalents, and shall not be restricted or limited by the foregoing detailed description.

1. A vitrectomy probe comprising:
a body defining a first diaphragm chamber;
a cutter comprising:
a tubular outer cutter coupled at a proximal end of the body; and
a tubular inner cutter disposed within the tubular outer cutter and movable therewithin;
an actuating mechanism comprising:
a flexible diaphragm coupled to the body, the first diaphragm chamber disposed adjacent to a first side of the flexible diaphragm; and
a fluidic amplifier comprising:
an interaction region;
a supply port in fluid communication with the interaction region, the supply port operable to introduce a power stream of fluid into the interaction region;
a first control port in fluid communication with the interaction region, the first control port operable to introduce a first control jet of fluid into the interaction region;
a vent port through which fluid is vented from the fluidic amplifier;
a splitter disposed at a distal end the interaction region and dividing the distal end of the interaction region into a first outlet and a second outlet, the first outlet fluidically coupled to the first diaphragm chamber and the second outlet fluidically coupled to the vent port; and
a first vent line fluidically coupled to the first outlet of the interaction region and fluidically coupled to the vent port.

2. The vitrectomy probe of claim 1, further comprising a spring positioned on a second side of the flexible diaphragm opposite the first side, the spring configured to apply a biasing force to the flexible diaphragm.

3. The vitrectomy probe of claim 1, wherein the splitter is offset from the supply port such that the splitter redirects a flow from the supply port into the first outlet.

4. The vitrectomy probe of claim 1, wherein the splitter is offset from the supply port such that the splitter redirects a flow from the supply port into the second outlet.

5. The vitrectomy probe of claim 1, wherein the interaction region comprises a sidewall shaped to promote attachment of the power stream to the sidewall.

6. The vitrectomy probe of claim 1, wherein the splitter is aligned with the supply port such that the splitter redirects a flow from the supply port substantially equally into the first outlet and the second outlet.

7. The vitrectomy probe of claim 1, further comprising:
a second diaphragm chamber disposed adjacent to a second side of the flexible diaphragm, the second outlet fluidically coupled to the second diaphragm chamber; and
a second vent line extending from the second outlet to the vent port.

8. The vitrectomy probe of claim 7, wherein the splitter is offset from the supply port such that the splitter redirects a flow from the supply port into the first outlet of the interaction region.

9. The vitrectomy probe of claim 7, wherein the interaction region comprises a sidewall shaped to promote attachment of the power stream to the sidewall.

10. The vitrectomy probe of claim 1, wherein vitrectomy probe further comprises:
a second control port fluidically coupled to the interaction region, the second control portion operable to introduce a second control jet into the interaction region; and
wherein the splitter is aligned with the supply port such that the splitter divides a power stream flow from the supply port substantially equally into the first outlet of the interaction region and the second outlet of the interaction region.

11. A method for actuating a cutter of a vitrectomy probe, the method comprising:
supplying a power stream to a fluidic amplifier disposed within the vitrectomy probe, a tubular inner cutter of the cutter disposed in a first position when the power stream is supplied to the fluidic amplifier; and
selectively supplying a control jet to the fluidic amplifier with such that when the control jet is supplied, the power stream is redirected from a first path to a second path within the fluidic amplifier to actuate the tubular inner cutter from the first position to a second position, and when the control jet is not supplied, the power stream is returned to the first path within the fluidic amplifier, causing the tubular inner cutter to return to the first position.

12. The method of claim 11, further comprising:
determining a desired cutting rate of the cutter;
configuring the fluidic amplifier such that the desired cutting rate of the cutter corresponds to a desired frequency of the control jet that is supplied to the fluidic amplifier; and
setting the desired cutting rate for cutter by setting the control jet to a desired frequency.

13. A vitrectomy probe comprising:
a body defining a first diaphragm chamber;
a tubular outer cutter coupled at a proximal end to the body;
an aspiration port formed in a distal end of the tubular outer cutter;
a tubular inner cutter disposed within the tubular outer cutter, the tubular inner cutter movable within the tubular outer cutter; and
an actuating mechanism operable to actuate the tubular inner cutter, the actuating mechanism housed in the body and comprising:
a flexible diaphragm coupled to the body, the first diaphragm chamber disposed adjacent to a first side of the flexible diaphragm; and
a fluidic oscillator comprising:
an interaction region;
a supply port in fluid communication with the interaction region, the supply port operable to introduce a power stream of fluid into the interaction region;
a vent port through which fluid is vented from the fluidic oscillator;
a first feedback channel offset from the interaction region by a first wall, the first feedback channel operable to redirect the power stream of fluid in a first direction;
a second feedback channel offset from the interaction region by a second wall, the second feedback channel operable to redirect the power stream in a second direction opposite the first direction;
a nozzle at the distal end of the interaction region;
a first outlet extending from the nozzle and fluidically coupled to the first diaphragm chamber;
a second outlet extending from the nozzle and fluidically coupled to the vent port; and
a first vent line fluidically coupled to the first outlet of the fluidic oscillator and fluidically coupled to the vent port.

14. The vitrectomy probe of claim 13, further comprising:
a second diaphragm chamber disposed on a second side of the flexible diaphragm opposite the first side; and
a second vent line extending from the second outlet to the vent port, wherein the first outlet of the fluidic oscillator is fluidically coupled to one of the first diaphragm chamber or the second diaphragm chamber; and
wherein the second outlet of the fluidic oscillator is fluidically coupled to the other of the first diaphragm chamber or the second diaphragm chamber.

15. The vitrectomy probe of claim 13, further comprising a spring abutting the flexible diaphragm along a second side of the flexible diaphragm opposite the first side.

16. The vitrectomy probe of claim 13, wherein the fluidic oscillator further comprises a splitter that separates the first outlet from the second outlet.
